(19) United States
(12) Patent Application Publication
(10) Pub. No.: US 2007/0111311 A1
(43) Pub. Date: May 17, 2007

(54) Plant cells and plants with increased tolerance to environmental stress

(75) Inventors: Piotr Puzio, Berlin (DE); Agnes Chardonnens, Berlin (DE); Ruoying Chen, Apex, NC (US); Pilar Puente, Durham, NC (US)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 11/250,779

(22) Filed: Oct. 14, 2005

Related U.S. Application Data
(63) Continuation-in-part of application No. PCT/US04/11887, filed on Apr. 15, 2004.

(30) Foreign Application Priority Data
Apr. 15, 2003 (EP) 03008079.0
Aug. 1, 2003 (EP) 03016671.4
Sep. 30, 2003 (EP) 03022226.9

(51) Int. Cl. C12N 15/82; C12N 15/29; C07H 21/02

(52) U.S. Cl. 435/419; 800/298; 800/285; 536/23.1; 530/370; 530/387.1

(57) ABSTRACT

This invention relates generally to transformed plant cells and plants comprising an inactivated or down-regulated gene resulting in increased tolerance and/or resistance to environmental stress as compared to non-transformed wild type cells and methods of producing such plant cells or plants. This invention further relates generally to transformed plant cells with altered metabolic activity compared to a corresponding non-transformed wild type plant cell, wherein the metabolic activity is altered by an inactivated or down-regulated gene and results in increased tolerance and/or resistance to an environmental stress as compared to a corresponding non transformed wild type plant cell, methods of producing, screening for and breeding such plant cells or plants and method of detecting stress in plants cells or plants.

This application is a continuation-in-part of international application PCT/US2004/011887 filed Apr. 15, 2004 which claims benefit to European application 03008079.0 filed Apr. 15, 2003, European application 03016671.4 filed Aug. 1, 2003, and European application 03022226.9 filed Sep. 30, 2003 each of which is incorporated by reference herein in its entirety for all useful purposes.

This invention relates generally to transformed plant cells and plants comprising an inactivated or down-regulated gene resulting in increased tolerance and/or resistance to environmental stress as compared to non-transformed wild type cells and methods of producing such plant cells or plants.

This invention further relates generally to transformed plant cells with altered metabolic activity compared to a corresponding non transformed wild type plant cell, wherein the metabolic activity is altered by an inactivated or down-regulated gene and results in increased tolerance and/or resistance to an environmental stress as compared to a corresponding non-transformed wild type plant cell, methods of producing, screening for and breeding such plant cells or plants and method of detecting stress in plants cells or plants.

In particular, this invention relates to transformed plant cells and plants comprising an inactivated or down-regulated gene resulting in increased tolerance and/or resistance to environmental stress, especially by altering the metabolic activity, as compared to non-transformed wild type cells and methods of producing such plant cells or plants.

Abiotic environmental stress, such as drought stress, salinity stress, heat stress, and cold stress, is a major limiting factor of plant growth and productivity (Boyer. 1982. Science 218, 443-448). Crop losses and crop yield losses of major crops such as rice, maize (corn) and wheat caused by these stresses represent a significant economic and political factor and contribute to food shortages in many underdeveloped and third-world countries.

Plants are typically exposed during their life cycle to conditions of reduced environmental water content. Most plants have evolved strategies to protect themselves against these conditions of low water or desiccation (drought) for short period of time. However, if the severity and duration of the drought conditions are too great, the effects on plant development, growth and yield of most crop plants are profound. Continuous exposure to drought causes major alterations in the plant metabolism. These great changes in metabolism ultimately lead to cell death and consequently yield losses.

Developing stress-tolerant and/or resistant plants is a strategy that has the potential to solve or mediate at least some of these problems (McKersie and Leshem, 1994. Stress and Stress Coping in Cultivated Plants, Kluwer Academic Publishers). However, traditional plant breeding strategies to develop new lines of plants that exhibit resistance (tolerance) to these types of stress are relatively slow and require specific resistant lines for crossing with the desired line. Limited germplasm resources for stress tolerance and incompatibility in crosses between distantly related plant species represent significant problems encountered in conventional breeding. Additionally, the cellular processes leading to drought, cold and salt tolerance and/or resistance are complex in nature and involve multiple mechanisms of cellular adaptation and numerous metabolic pathways (McKersie and Leshem, 1994. Stress and Stress Coping in Cultivated Plants, Kluwer Academic Publishers). This multi-component nature of stress tolerance and/or resistance has not only made breeding for tolerance and/or resistance largely unsuccessful, but has also limited the ability to genetically engineer stress tolerance plants using biotechnological methods.

Drought, heat, cold and salt stress have a common theme important for plant growth and that is water availability. Plants are exposed during their entire life cycle to conditions of reduced environmental water content. Most plants have evolved strategies to protect themselves against lack of water. However, if the severity and duration of the drought conditions are too great, the effects on plant development, growth and yield of most crop plants are profound. Since high salt content in some soils result in less available water for cell intake, its effect is similar to those observed under drought conditions. Likewise, under freezing temperatures, plant cells loose water as a result of ice formation that starts in the apoplast and withdraws water from the symplast (McKersie and Leshem, 1994. Stress and Stress Coping in Cultivated Plants, Kluwer Academic Publishers). Commonly, a plant's molecular response mechanisms to each of these stress conditions are the same.

The results of current research indicate that drought tolerance and/or resistance is a complex quantitative trait and that no real diagnostic marker is available yet. High salt concentrations or dehydration may cause damage at the cellular level during drought stress but the precise injury is not entirely clear (Bray, 1997. Trends Plant Sci. 2, 48-54). This lack of a mechanistic understanding makes it difficult to design a transgenic approach to improve drought tolerance and/or resistance. However, an important consequence of damage may be the production of reactive oxygen radicals that cause cellular injury, such as lipid peroxidation or protein and nucleic acid modification. Details of oxygen free radical chemistry and their reaction with cellular components such as cell membranes have been described (McKersie and Leshem, 1994. Stress and Stress Coping in Cultivated Plants, Kluwer Academic Publishers).

There are numerous sites of oxygen activation in the plant cell, which are highly controlled and tightly coupled to prevent release of intermediate products (McKersie and Leshem, 1994. Stress and Stress Coping in Cultivated Plants, Kluwer Academic Publishers). Under abiotic stress situations, it is likely that this control or coupling breaks down and the process “dysfunctions” leaking activated oxygen. These uncoupling events are not detrimental provided that they are short in duration and that the oxygen scavenging systems are able to detoxify the various forms of activated oxygen. If the production of activated oxygen exceeds the plant's capacity to detoxify it, deleterious degenerative reactions occur. At the subcellular level, disintegration of membranes and aggregation of proteins are typical symptoms. Therefore it is the balance between the production and the scavenging of activated oxygen that is critical to the maintenance of active growth and metabolism of the plant and overall environmental (abiotic) stress tolerance and/or resistance.

Preventing or diminishing the accumulation of oxygen free radicals in response to drought is a potential way to engineer tolerance (Allen, 1995. Plant Physiol. 107, 1049-1054). Overexpression of antioxidant enzymes or ROS-scavenging enzymes is one possibility for the induction of functional detoxification systems. For example, transgenic alfalfa plants expressing Mn-superoxide dismutase tend to have reduced injury after water-deficit stress (McKersie et al., 1996. Plant Physiol. 111, 1177-1181). These same transgenic plants have increased biomass production in field trials (McKersie et al., 1999. Plant Physiology, 119: 839-847; McKersie et al., 1996. Plant Physiol. 111, 1177-1181). Transgenic plants that overproduce osmolytes such as mannitol, fructans, proline or glycine-betaine also show increased resistance to some forms of abiotic stress and it is proposed that the synthesized osmolytes act as ROS scavengers (Tarczynski. et al. 1993 Science 259, 508-510; Sheveleva, et al. 1997. Plant Physiol. 115, 1211-1219).

It is the object of this invention to identify new, unique genes capable of conferring stress tolerance to plants upon inactivation or down-regulation of genes.

It is further object of this invention to identify, produce and breed new, unique stress tolerant and/or resistant plant cells or plants and methods of inducing and detecting stress tolerance and/or resistance in plants or plant cells. It is a further object to identify new methods to detect stress tolerance and/or resistance in plants or plant cells.

It is also the object of this invention to identify new, unique genes capable of conferring stress tolerance to plants, which is preferably achieved by altering metabolic activity, upon inactivated or down-regulated genes

The present invention provides a transformed plant cell with altered metabolic activity compared to a corresponding non transformed wild type plant cell, wherein the metabolic activity is altered by an inactivated or down-regulated gene and results in increased tolerance and/or resistance to an environmental stress as compared to a corresponding non-transformed wild type plant cell.

As used herein, the term “metabolite” refers to intermediate substances, preferably such of low molecular weight, which occur during anabolism and catabolism in a cell or plant.

The term “altered metabolic activity” refers to the change (increase oe decrease) of the amount, concentration or activity (meaning here the effective concentration for the purposes of chemical reactions and other mass action) of a metabolite in a specific volume relative to a corresponding volume (e.g. in an organism, a tissue, a cell or a cell compartment) of a control, reference or wild type, measured for example by one of the methods described herein below, which is changed (increased or decreased) as compared to a corresponding non transformed wild type plant cell.

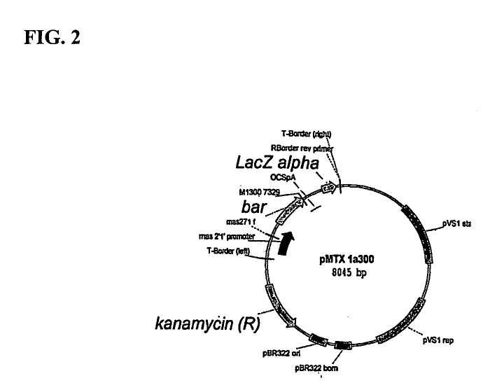

As used herein, the term “inactivated or down-regulated gene” means the transgenic reduction or deletion of the expression of nucleic acid of FIG. 1a, 1b, 1c or 1d leading to an altered metabolic activity and which results in increased tolerance and/or resistance to an environmental stress as compared to a corresponding non-transformed wild type plant cell.

In the transgenic plant cell of the invention, the reduction or deletion of the expression of said nucleic acid results in increased tolerance to an environmental stress, which is preferably achieved by altering metabolic activity, as compared to a corresponding non-transformed wild type plant cell. Herein, the environmental stress is selected from the group consisting of salinity, drought, temperature, metal, chemical, pathogenic and oxidative stresses, or combinations thereof, preferably drought and/or temperature.

The term “expression” refers to the transcription and/or translation of a codogenic gene segment or gene. As a rule, the resulting product is an mRNA or a protein. However, expression products can also include functional RNAs such as, for example, antisense, nucleic acids, tRNAs, snRNAs, rRNAs, RNAi, siRNA, ribozymes etc. Expression may be systemic, local or temporal, for example limited to certain cell types, tissues organs or time periods.

Unless otherwise specified, the terms “polynucleotides”, “nucleic acid” and “nucleic acid molecule” are interchangeably in the present context. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refers to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and single-stranded DNA and RNA. They also include known types of modifications, for example, methylation, “caps”, substitutions of one or more of the naturally occurring nucleotides with an analog. Preferably, the DNA or RNA sequence of the invention comprises a coding sequence encoding the herein defined polypeptide.

A “coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

For the purposes of the invention, as a rule the plural is intended to encompass the singular and vice versa.

The terms “reduction”, “decrease” or “deletion” relate to a corresponding change of a property in an organism, a part of an organism such as a tissue, seed, root, leave, flower etc. or in a cell. Under “change of a property” it is understood that the activity, expression level or amount of a gene product or the metabolite content is changed in a specific volume or in a specific amount of protein relative to a corresponding volume or amount of protein of a control, reference or wild type. Preferably, the overall activity in the volume is reduced, decreased or deleted in cases if the reduction, decrease or deletion is related to the reduction, decrease or deletion of an activity of a gene product, independent whether the amount of gene product or the specific activity of the gene product or both is reduced, decreased or deleted or whether the amount, stability or translation efficacy of the nucleic acid sequence or gene encoding for the gene product is reduced, decreased or deleted.

The terms “reduction”, “decrease” or “deletion” include the change of said property in only parts of the subject of the present invention, for example, the modification can be found in compartment of a cell, like an organelle, or in a part of a plant, like tissue, seed, root, leave, flower etc. but is not detectable if the overall subject, i.e. complete cell or plant, is tested. Preferably, the “reduction”, “decrease” or “deletion” is found cellular, thus the term “reduction, decrease or deletion of an activity” or “reduction, decrease or deletion of a metabolite content” relates to the cellular reduction, decrease or deletion compared to the wild type cell. In addition the terms “reduction”, “decrease” or “deletion” include the change of said property only during different growth phases of the organism used in the inventive process, for example the reduction, decrease or deletion takes place only during the seed growth or during blooming. Furthermore the terms include a transitional reduction, decrease or deletion for example because the used RNAi is not stable integrated in the genome of the organism and has therefore only a transient effect.

Accordingly, the term “reduction”, “decrease” or “deletion” means that the specific activity of an enzyme or other protein or regulatory RNA as well as the amount of a compound or metabolite, e.g. of a polypeptide, a nucleic acid molecule or the fine chemical of the invention or an encoding mRNA or DNA, can be reduced, decreased or deleted in a volume.

The terms “wild type”, “control” or “reference” are exchangeable and can be a cell or a part of organisms such as an organelle or tissue, or an organism, in particular a microorganism or a plant, which was not modified or treated according to the herein described process according to the invention. Accordingly, the cell or a part of organisms such as an organelle or a tissue, or an organism, in particular a microorganism or a plant used as wild type, control or reference corresponds to the cell, organism or part thereof as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property.

Preferably, any comparison is carried out under analogous conditions. The term “analogous conditions” means that all conditions such as, for example, culture or growing conditions, assay conditions (such as buffer composition, temperature, substrates, pathogen strain, concentrations and the like) are kept identical between the experiments to be compared.

The “reference”, “control”, or “wild type” is preferably a subject, e.g. an organelle, a cell, a tissue, an organism, in particular a plant or a microorganism, which was not modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-” “control-” or “wild type-”-organelle, -cell, -tissue or -organism, in particular plant or microorganism, relates to an organelle, cell, tissue or organism, in particular plant or microorganism, which is nearly genetically identical to the organelle, cell, tissue or organism, in particular microorganism or plant, of the present invention or a part thereof preferably 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is preferably a subject, e.g. an organelle, a cell, a tissue, an organism, which is genetically identical to the organism, cell organelle used according to the process of the invention except that nucleic acid molecules or the gene product encoded by them are changed according to the inventive process.

Preferably, the reference, control or wild type differs form the subject of the present invention only in the cellular activity of the polypeptide or RNA of the invention, e.g. as result of a reduction, decrease or deletion in the level of the nucleic acid molecule of the present invention or a reduction, decrease or deletion of the specific activity of the polypeptide or RNA of the invention, e.g. by or in the expression level or activity of protein or RNA that means its biological activity and/or its biochemical or genetical causes.

The term “expression” means the transcription of a gene into structural RNA (rRNA, tRNA, miRNA) or messenger RNA (mRNA) with the subsequent translation of the latter into a protein. Experimentally, expression can be detected by e.g. Northern, qRT PCR, transcriptional run-on assays or Western blotting and other immuno assays. As consequence of the reduction, decrease or deletion of the expression that means as consequence of the reduced, decreased or deleted transcription of a gene a related phenotypic trait appears such as the enhanced or increased stress tolerance.

Accordingly, preferred reference subject is the starting subject of the present process of the invention. Preferably, the reference and the subject matter of the invention are compared after standardization and normalization, e.g. to the amount of total RNA, DNA, or Protein or activity or expression of reference genes, like housekeeping genes, such as ubiquitin.

A series of mechanisms exists via which a modification in the polypeptide of the invention can directly or indirectly affect stress tolerance. For example, the molecule number or the specific activity of the polypeptide of the invention or the number of expression of the nucleic acid molecule of the invention may be reduced, decreased or deleted. However, it is also possible to reduce, decrease or delete the expression of the gene which is naturally present in the organisms, for example by modifying the regulation of the gene, or by reducing or decreasing the stability of the mRNA or of the gene product encoded by the nucleic acid molecule of the invention.

This also applies analogously to the combined reduction, decrease or deletion of the expression of the nucleic acid molecule of the present invention or its gene product together with the manipulation of further activities such as enzymes which confer stress tolerance.

The reduction, decrease, deletion or modulation according to this invention can be constitutive, e.g. due to a stable permanent transgenic expression or to a stable mutation in the corresponding endogenous gene encoding the nucleic acid molecule of the invention or to a modulation of the expression or of the behaviour of a gene conferring the expression of the polypeptide of the invention, or transient, e.g. due to an transient transformation, a transiently active promoter or temporary addition of a modulator such as an antagonist or inductor, e.g. after transformation with a inducible construct carrying a double-stranded RNA nucleic acid molecule, an antisense nucleic acid molecule, a ribozyme of the invention etc. under control of a inducible promoter and adding the inducer, e.g. tetracycline or as described herein below.

The reduction, decrease or deletion in activity amounts preferably by at least 10%, preferably by at least 30% or at least 60%, especially preferably by at least 70%, 80%, 85%, 90% or more, very especially preferably are at least 95%, more preferably are at least 99% or more in comparison to the control, reference or wild type. Most preferably the reduction, decrease or deletion in activity amounts to 100%.

In this context, inactivation means that the enzymatic or biological activity of the polypeptides encoded is no longer detectable in the organism or in the cell such as, for example, within the plant or plant cell. For the purposes of the invention, downregulation (=reduction) means that the enzymatic or biological activity of the polypeptides encoded is partly or essentially completely reduced in comparison with the activity of the untreated organism. This can be achieved by different cell-biological mechanisms. In this context, the activity can be downregulated in the entire organism or, in the case of multi-celled organisms, in individual parts of the organism, in the case of plants for example in tissues such as the seed, the leaf, the root or other parts. In this context, the enzymatic activity or biological activity is reduced by at least 10%, advantageously at least 20%, preferably at least 30%, especially preferably at least 40%, 50% or 60%, very especially preferably at least 70%, 80%, 85% or 90% or more, very especially preferably are at least 95%, more preferably are at least 99% or more in comparison to the control, reference or wild type. Most preferably the reduction, decrease or deletion in activity amounts to 100%.

Various strategies for reducing the quantity (=expression), the activity or the function of proteins encoded by the nucleic acids or the nucleic acid sequences itself according to the invention are encompassed in accordance with the invention. The skilled worker will recognize that a series of different methods are available for influencing the quantity of a protein, the activity or the function in the desired manner.

The term “biological activity” means the biological function of the protein of the invention. In contrast to the term “biological activity” the term “activity” means the increase in the production of the compound produced by the inventive process. The term “biological activity” preferably refers to the enzymatic function, transporter carrier function, DNA-packaging function, heat shock protein function, recombination protein function, beta-galactosidase function, Serine/threonine-protein kinase CTR1 function, lipase function, enoyl-CoA hydratase function, UDP-glucose glucosyltransferase function, cell division protein function, flavonol synthase function, tracylglycerol lipase, MADS-box protein function, pectinesterase function, pectin metylesterase function, calcium transporting ATPase function, protein kinase function, lysophospholipase function, Chlorophyll A-B binding proteins function, Ca2+-transporting ATPase-like protein function, peroxidase function, disease resistance RPP5 like protein function, or regulatory function of a peptide or protein in an organism, a tissue, a cell or a cell compartment. Suitable substrates are low-molecular-weight compounds and also the protein interaction partners of a protein. The term “reduction” of the biological function refers, for example, to the quantitative reduction in binding capacity or binding strength of a protein for at least one substrate in an organism, a tissue, a cell or a cell compartment—for example by one of the methods described herein below—in comparison with the wild type of the same genus and species to which this method has not been applied, under otherwise identical conditions (such as, for example, culture conditions, age of the plants and the like). Reduction is also understood as meaning the modification of the substrate specificity as can be expressed for example, by the kcat/Km value. In this context, a reduction of the function of at least 10%, advantageously of at least 20%, preferably at least 30%, especially preferably of at least 40%, 50% or 60%, very especially preferably of at least 70%, 80%, 90% or 95%, in comparison with the untreated organism is advantageous. A particularly advantageous embodiment is the inactivation of the function. Binding partners for the protein can be identified in the manner with which the skilled worker is familiar, for example by the yeast 2-hybrid system.

A modification, i.e. a decrease, can be caused by endogenous or exogenous factors. For example, a decrease in activity in an organism or a part thereof can be caused by adding a chemical compound such as an antagonist to the media, nutrition, soil of the plants or to the plants themselves.

The transformed plant cells are compared to the corresponding non-transformed wild type of the same genus and species under otherwise identical conditions (such as, for example, culture conditions, age of the plants and the like). In this context, a change of at least 10%, advantageously of at least 20%, preferably at least 30%, especially preferably of at least 40%, 50% or 60%, very especially preferably of at least 70%, 80%, 90%, 95% or even 100% or more, in comparison with the non-transformed organism is advantageous.

Preferably the change in metabolite concentration of the transformed plant cells is the changed compared to the corresponding non-transformed wild type. Preferably the change in metabolite concentration is measured by HPLC and calculated by dividing the peak height or peak area of each analyte (metabolite) through the peak area of the respective internal standards. Data is normalized using the individual sample fresh weight. The resulting values are divided by the mean values found for wild type plants grown under control conditions and analysed in the same sequence, resulting in the so-called ratios, which represent values independent of the analytical sequence. These ratios indicate the behavior of the metabolite concentration of the transformed plants in comparison to the concentration in the wild type control plants.

According to this method, the change in at least one metabolite concentration of the transformed plant cells compared to the corresponding non-transformed wild type is at least 10%, advantageously of at least 20%, preferably at least 40%, 60% or 80%, especially preferably of at least 90%, 100% or 200%, very especially preferably of at least 300%, 350%, 400%, 500%, 600%, 800%, 1000% or more.

Data significance can be determinated by all statistical methods known by a person skilled in the art, preferably by a t-test, more preferably by the student t-test.

In a preferred embodiment of the invention, the altered metabolic activity also refers to metabolites that, compared to a corresponding non transformed wild type plant cell, are not produced after transformation or are only produced after transformation or the production of said metabolite is increased.

More preferred the concentration of at least one metabolite is reduced, most preferred the concentration of at least one metabolite is zero, or the concentration of at least one metabolite is increased, compared to a corresponding non transformed wild type plant cell and calculated according to the above described method.

Preferred metabolites of the invention are 2,3-dimethyl-5-phytylquinol or 2-hydroxy-palmitic acid or 3,4-dihydroxyphenylalanine (=dopa) or 3-hydroxy-palmitic acid or 5-oxoproline or alanine or alpha linolenic acid (c18:3 (c9, c12, c15)) or alpha-tocopherol or aminoadipic acid or anhydroglucose or arginine or aspartic acid or beta-apo-8′ carotenal or beta-carotene or beta-sitosterol or beta-tocopherol or (delta-7-cis,10-cis)-hexadecadienic acid or hexadecatrienic acid or margaric acid or delta-15-cis-tetracosenic acid or ferulic acid or campesterol or cerotic acid (c26:0) or citrulline or cryptoxanthine or eicosenoic acid (20:1) or fructose or fumarate or galactose or gamma-aminobutyric acid or gamma-tocopherol or gluconic acid or glucose or glutamic acid or glutamine or glycerate or glycerinaldehyd or glycerol or glycerol-3-phosphate or glycine or homoserine or inositol or isoleucine or iso-maltose or isopentenyl pyrophosphate or leucine or lignoceric acid (c24:0) or linoleic acid (c18:2 (c9, c12)) or luteine or lycopene or malate or mannose or methionine or methylgalactofuranoside or methylgalactopyranoside or methylgalactopyranoside or palmitic acid (c16:0) or phenylalanine or phosphate or proline or putrescine or pyruvat or raffinose or ribonic acid or serine or shikimate or sinapine acid or stearic acid (c18:0) or succinate or sucrose or threonine or triacontanoic acid or tryptophane or tyrosine or ubichinone or udp-glucose or valine or zeaxanthine.

Metabolic activity may also be altered concerning one or more derivates of one or more of the above metabolites.

Preferably metabolic activity is altered concerning one or more metabolites selected from the group consisting of all of the above metabolites.

Alternatively metabolic activity may be altered concerning one or more metabolites selected from the group consisting of mannose, inositol, phosphate, aspartic acid, isoleucine, leucine, gamma-aminobutyric acid, glycerinaldehyd, sucrose, campesterol, valine, beta-tocopherol, ubichinone, palmitic acid (c16:0), 2-hydroxy-palmitic acid, 2,3-dimethyl-5-phytylquinol, beta-carotene, alpha-linolenic acid (c18:3 (c9, c12, c15)), lycopene.

Alternatively metabolic activity may be altered concerning one or more metabolites selected from the group consisting of methylgalactofuranoside, beta-sitosterol, delta-15-cis-tetracosenic acid (c24:1 me), margaric acid (c17:0 me), stearic acid (c18:0), methylgalactopyranoside, gamma-tocopherol, linoleic acid (c18:2 (c9, c12)), hexadecatrienic acid (c16:3 me), shikimate, raffinose, glutamic acid, glutamine, udp-glucose, proline, threonine, isopentenyl pyrophosphate, 5-oxoproline, ferulic acid, sinapine acid.

Alternatively metabolic activity may be altered concerning one or more metabolites selected from the group consisting of tryptophane, citrulline, serine, alanine, glycerate, arginine, 3-hydroxy-palmitic acid, putrescine, 3,4-dihydroxyphenylalanine (=dopa), alpha-tocopherol, aminoadipic acid, anhydroglucose, beta-apo-8′ carotenal, delta-7-cis,10-cis-hexadecadienic acid (c16:2 me), cerotic acid (c26:0), cryptoxanthine, eicosenoic acid (20:1), fructose, fumarate.

Alternatively metabolic activity may be altered concerning one or more metabolites-selected from the group consisting of galactose, gluconic acid, glucose, glycerol, glycerol-3-phosphate, glycine, homoserine, iso-maltose, lignoceric acid (c24:0), luteine, malate, triacontanoic acid, methionine, phenylalanine, pyruvate, ribonic acid, succinate, tyrosine, zeaxanthine.

In the invention inactivation or down-regulation of a gene in the plant cell results in altered metabolic activity as compared to a corresponding non-transformed wild type plant cell. One preferred wild type plant cell is a non-transformed Arabidopsis plant cell. An example here is the Arabidopsis wild type C24 (Nottingham Arabidopsis Stock Centre, UK; NASC Stock N906).

Other preferred wild type plant cells are a non-transformed from plants selected from the group consisting of maize, wheat, rye, oat, triticale, rice, barley, soybean, peanut, cotton, rapeseed, canola, manihot, pepper, sunflower, flax, borage, safflower, linseed, primrose, rapeseed, turnip rape, tagetes, solanaceous plants, potato, tobacco, eggplant, tomato, Vicia species, pea, alfalfa, coffee, cacao, tea, Salix species, oil palm, coconut, perennial grass and forage crops.

More preferred wild type plant cells are a non-transformed Linum plant cell, preferably Linum usitatissimum, more preferably the variety Brigitta, Golda, Gold Merchant, Helle, Juliel, Olpina, Livia, Marlin, Maedgold, Sporpion, Serenade, Linus, Taunus, Lifax or Liviola, a non-transformed Heliantus plant cell, preferably Heliantus annuus, more preferably the variety Aurasol, Capella, Flavia, Flores, Jazzy, Palulo, Pegasol, PIR64A54, Rigasol, Sariuca, Sideral, Sunny, Alenka, Candisol or Floyd, or a non-transformed Brassica plant cell, preferably Brassica napus, more preferably the variety Dorothy, Evita, Heros, Hyola, Kimbar, Lambada, Licolly, Liconira, Licosmos, Lisonne, Mistral, Passat, Serator, Siapula, Sponsor, Star, Caviar, Hybridol, Baical, Olga, Lara, Doublol, Karola, Falcon, Spirit, Olymp, Zeus, Libero, Kyola, Licord, Lion, Lirajet, Lisbeth, Magnum, Maja, Mendel, Mica, Mohican, Olpop, Ontarion, Panthar, Prinoe, Pronio, Susanna, Talani, Titan, Transfer, Wiking, Woltan, Zeniah, Artus, Contact or Smart.

Inactivation or down-regulation of a gene is advantageous since no new gene must be introduced to achieve the altered metabolic activity resulting in increased tolerance and/or resistance to environmental stress. Only an endogenous gene is hindered in its expression.

The inactivated or down-regulated gene or genes directly or indirectly influence the stress tolerance of plants, preferably the metabolic activity of the transformed plant cells. Preferably they influence the activity of the above metabolites.

Stress tolerance, conferred preferably by altered metabolic activity may be conferred by one or more inactivated or down-regulated genes encoded by one or more nucleic acid sequences selected from the group consisting of
a) nucleic acid molecule encoding on of the polypeptides shown in FIG. 1a, 1b, 1c or 1d;
b) nucleic acid molecule comprising at least one of the nucleic acid molecules shown in FIG. 1a, 1b, 1c or 1d;
c) nucleic acid molecule comprising a nucleic acid sequence, which, as a result of the degeneracy of the genetic code, can be derived from a polypeptide sequence depicted in FIG. 1a, 1b, 1c or 1d;
d) nucleic acid molecule encoding a polypeptide having at least 50% identity with the amino acid sequence of the polypeptide encoded by the nucleic acid molecule of (a) to (c) and having the biological activity represented by protein of FIG. 1a, 1b, 1c or 1d;
e) nucleic acid molecule encoding a polypeptide which is isolated with the aid of monoclonal antibodies against a polypeptide encoded by one of the nucleic acid molecules of (a) to (d) and having the biological activity represented by the protein of FIG. 1a, 1b, 1c or 1d;
f) nucleic acid molecule which is obtainable by screening a suitable nucleic acid library under stringent hybridization conditions with a probe comprising one of the sequences of the nucleic acid molecule of (a) or (b) or with a fragment thereof having at least 15 nucleotides of the nucleic acid molecule characterized in (a) to (c) and encoding a polypeptide having the biological activity represented by a protein whose reduction or deletion results in increased tolerance and/or resistance to an environmental stress

or which comprises a sequence which is complementary thereto.

For the purpose of the present invention the term FIG. 1a, 1b, 1c or 1d involves and means the SEQ ID NO: 1 to 112 and 114 to 283 and the term FIG. 2 means SEQ ID NO: 113.

Particularly, the term FIG. 1a means SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 and/or 90.

The term FIG. 1b means SEQ ID NO: 91, 92, 93, 94, 95, 96, 97, 98, 99, 100, 101, 102, 103, 104, 105, 106, 107, 108, 109, 110 and/or 111.

The term FIG. 1c means SEQ ID NO: 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or

115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283.

The term FIG. 1d means SEQ ID NO 116, 130, 132, 146, 162, 177, 210, 212, 220, 222, 242, and/or 279 and/or 117, 131, 133, 147, 163, 178, 211, 213, 221, 223, 243 and/or 280.

More precisely, when polypeptides or proteins according to FIG. 1a are mentioned, then the SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 are meant;

when polypeptides or proteins according to FIG. 1b are mentioned, then the SEQ ID NO 92, 94, 96, 98, 100, 102, 104, 106, 108, 110 and/or 112 are meant; when polypeptides or proteins according to FIG. 1c are mentioned, then the SEQ ID NO 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 are meant;

when polypeptides or proteins according to FIG. 1d are mentioned, then the SEQ ID NO 117, 131, 133, 147, 163, 178, 211, 213, 221, 223, 243 and/or 280 are meant.

More precisely, when polynucleotides or nucleic acid molecules according to FIG. 1a are mentioned, then the SEQ ID NO 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 are meant;

when polynucleotides or nucleic acid molecules according to FIG. 1b are mentioned, then the SEQ ID NO 91, 93, 95, 97, 99, 101, 103, 105, 107, 109 and/or 111 are meant;

when polynucleotides or nucleic acid molecules according to FIG. 1c are mentioned, then the SEQ ID NO 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 are meant;

when polynucleotides or nucleic acid molecules according to FIG. 1d are mentioned, then the SEQ ID NO 116, 130, 132, 146, 162, 177, 210, 212, 220, 222, 242, and/or 279 are meant.

With the present invention it is possible to identify the genes encoded by a nucleic acid sequence selected from the group consisting of sequences shown in FIG. 1a, 1 b, 1c or 1d and/or homologs thereof in target plants, especially crop plants, and then inactivate or down-regulate the corresponding gene to achieve an increased tolerance and/or resistance to environmental stress (preferably by the altered metabolic activity). Consequently the invention is not limited to a specific plant.

It is further possible to detect environmental stress in plant cells or plants by screening the plant cells for altered metabolic activity as compared to non-stress conditions. This allows for monitoring of stress levels in plants, even when no symptoms are visible. Therefore counter action can be taken earlier and e.g. crop losses minimized by timely watering.

It is also within the scope of the invention to screen plant cells or plants for increased tolerance and/or resistance to environmental stress by screening the plant cells under stress conditions for altered metabolic activity as compared to non-stress conditions. This allows selection of plants with increased tolerance and/or resistance to environmental stress without the identification of genes or visual symptoms.

With the invention it is further possible to breed plant cells or plants towards increased tolerance and/or resistance to environmental stress by screening the plant cells under stress conditions for altering metabolic activity as compared to non-stress conditions and selecting those with increased tolerance and/or resistance to environmental stress. The screening for metabolite activity is faster and easier than e.g. screening for genes.

Screening is well known to those skilled in the art and generally refers to the search for a particular attribute or trait. In the invention this trait in a plant or plant cell is preferably the concentration of a metabolite, especially preferred the concentration of the above metabolites. The methods and devices for screening are familiar to those skilled in the art and include GC (gas chromatography), LC (liquid chromatography), HPLC (high performance (pressure) liquid chromatography), MS (mass spectrometry), NMR (nuclear magnetic resonance) spectroscopy, IR (infra red) spectroscopy, photometric methods etc and combinations of these methods.

Breeding is also customary knowledge for those skilled in the art. It is understood as the directed and stable incorporation of a particular attribute or trait into a plant or plant cell.

The various breeding steps are characterized by well-defined human intervention such as selecting the lines to be crossed, directing pollination of the parental lines, or selecting appropriate progeny plants. Different breeding measures can be taken, depending on the desired properties. All the techniques are well known by a person skilled in the art and include for example, but are not limited to hybridization, inbreeding, backcross breeding, multiline breeding, variety blend, interspecific hybridization, aneuploid techniques, etc. Hybridization techniques also can include the sterilization of plants to yield male or female sterile plants by mechanical, chemical, or biochemical means. Cross pollination of a male sterile plant with pollen of a different line assures that the genome of the male sterile but female fertile plant will uniformly obtain properties of both of the parental lines. The transgenic seeds and plants according to the invention can therefor be used for the breeding of improved plant lines, which can increase the effectiveness of conventional methods such as herbicide or pesticide treatment or which allow one to dispense with said methods due to their modified genetic properties. Alternatively new crops with improved stress tolerance, preferably drought and temperature, can be obtained, which, due to their optimized genetic “equipment”, yield harvested product of better quality than products that were not able to tolerate comparable adverse developmental conditions.

Environmental stress includes but is not limited to salinity, drought, temperature, metal, chemical, pathogenic and oxidative stress, or combinations thereof, preferably drought and/or temperature.

As used herein, the term “environmental stress” refers to any sub-optimal growing condition and includes, but is not limited to, sub-optimal conditions associated with salinity, drought, temperature, metal, chemical, pathogenic and oxidative stresses, or combinations thereof. In preferred embodiments, environmental stress may be salinity, drought, heat, or low temperature, or combinations thereof, and in particular, may be low water content or low temperature. Wherein drought-stress means any environmental stress which leads to a lack of water in plants or reduction of water supply to plants; wherein low temperature stress means freezing of plants below +4° C. as well as chilling of plants below 15° C. and wherein high temperature stress means for example a temperature above 35° C. The range of stress and stress response depends on the different plants which are used for the invention, i.e. it differs for example between a plant such as wheat and a plant such as Arabidopsis. It is also to be understood that as used in the specification and in the claims, “a” or “an” can mean one or more, depending upon the context in which it is used. Thus, for example, reference to “a cell” means that at least one cell may be utilized.

The invention also provides a transformed plant cell with one or more nucleic acid sequences homologous to one or more of sequences of FIG. 1a, 1b, 1c or 1d, wherein the plant is selected from the group comprised of maize, wheat, rye, oat, triticale, rice, barley, soybean, peanut, cotton, rapeseed, canola, manihot, pepper, sunflower, flax, borage, safflower, linseed, primrose, rapeseed, turnip rape, tagetes, solanaceous plants, potato, tobacco, eggplant, tomato, Vicia species, pea, alfalfa, coffee, cacao, tea, Salix species, oil palm, coconut, perennial grass, forage crops and Arabidopsis thaliana.

The present invention further provides a transgenic plant cell with an inactivated or down-regulated gene selected from the group comprising sequences of FIG. 1a, 1b, 1c or 1d and/or homologs thereof, preferably Brassica napus, Glycine max or Oryza sativa.

Furthermore it is possible to identify the genes encoded by a nucleic acid sequence selected from the group consisting of sequences of FIG. 1a, 1b, 1c or 1d and/or homologs thereof in target plants, especially crop plants, and then inactivate or down-regulate the corresponding gene to achieve increased tolerance and/or resistance to environmental stress. Consequently the invention is not limited to a specific plant.

The invention also provides a transformed plant cell with a nucleic acid sequence homologous to one of sequences of FIG. 1a, 1b, 1c or 1d, wherein the plant is selected from the group comprised of maize, wheat, rye, oat, triticale, rice, barley, soybean, peanut, cotton, rapeseed, canola, manihot, pepper, sunflower, flax, borage, safflower, linseed, primrose, rapeseed, turnip rape, tagetes, solanaceous plants, potato, tobacco, eggplant, tomato, Vicia species, pea, alfalfa, coffee, cacao, tea, Salix species, oil palm, coconut, perennial grass, forage crops and Arabidopsis thaliana.

Also the invention provides a transformed plant cell, wherein the nucleic acid or acids are at least about 30%, especially at least about 50% homologous to sequences of FIG. 1a, 1b, 1c or 1d.

According to the invention the transformed plant cell may be derived from a monocotyledonous or a dicotyledonous plant.

The monocotyledonous or a dicotyledonous plant may be selected from the group comprised of maize, wheat, rye, oat, triticale, rice, barley, soybean, peanut, cotton, rapeseed, canola, manihot, pepper, sunflower, flax; borage, safflower, linseed, primrose rapeseed, turnip-Tape, tagetes, solanaceous plants, potato, tobacco, eggplant, tomato, Vicia species, pea, alfalfa, coffee, cacao, tea, Salix species, oil palm, coconut, perennial grass, forage crops and Arabidopsis thaliana.

The transformed plant cell may be derived from a gymnosperm plant and can preferably be selected from the group of spruce, pine and fir.

The invention also provides a transformed plant generated from said plant cell and which is a monocot or dicot plant.

The transformed plant may be selected from the group comprised of maize, wheat, rye, oat, triticale, rice, barley, soybean, peanut, cotton, rapeseed, canola, manihot, pepper, sunflower, flax, borage, safflower, linseed, primrose, rapeseed, turnip rape, tagetes, solanaceous plants, potato, tobacco, eggplant, tomato, Vicia species, pea, alfalfa, coffee, cacao, tea, Salix species, oil palm, coconut, perennial grass, forage crops and Arabidopsis thaliana.

Preferably the transformed, plant generated from said plant cell is a gymnosperm plant, more preferred a plant selected from the group consisting of spruce, pine and fir.

The invention not only deals with plants but also with an agricultural product produced by any of the described transformed plants, plant parts such as leafs, petal, anther, roots, tubers, stems, buds, flowers or especially seeds produced by said transformed plant, which are at least genetically heterozygous, preferably homozygous for a gene or its homolog, that when inactivated or down-regulated confers an increased tolerance and/or resistance to environmental stress as compared to a wild type plant.

Homologs of the aforementioned sequences can be isolated advantageously from yeast, fungi, viruses, algae bacteria, such as Acetobacter (subgen. Acetobacter) aceti; Acidithiobacillus ferrooxidans; Acinetobacter sp.; Actinobacillus sp; Aeromonas salmonicida; Agrobacterium tumefaciens; Aquifex aeolicus; Arcanobacterium pyogenes; Aster yellows phytoplasma; Bacillus sp.; Bifidobacterium sp.; Borrelia burgdorferi; Brevibacterium linens; Brucella melitensis; Buchnera sp.; Butyrivibrio fibrisolvens; Campylobacter jejuni; Caulobacter crescentus; Chlamydia sp.; Chlamydophila sp.; Chlorobium limicola; Citrobacter rodentium; Clostridium sp.; Comamonas testosteroni; Corynebacterium sp.; Coxiella burnetii; Deinococcus radiodurans; Dichelobacter nodosus; Edwardsiella ictaluri; Enterobacter sp.; Erysipelothrix rhusiopathiae; Escherichia coli; Flavobacterium sp.; Francisella tularensis; Frankia sp. Cpl1; Fusobacterium nucleatum; Geobacillus stearothermophilus; Gluconobacter oxydans; Haemophilus sp.; Helicobacter pylori; Klebsiella pneumoniae; Lactobacillus sp.; Lactococcus lactis; Listeria sp.; Mannheimia haemolytica; Mesorhizobium loti; Methylophaga thalassica; Microcystis aeruginosa; Microscilla sp. PRE1; Moraxella sp. TA144; Mycobacterium sp.; Mycoplasma sp.; Neisseria sp.; Nitrosomonas sp.; Nostoc sp. PCC 7120; Novosphingobium aromaticivorans; Oenococcus oeni; Pantoea citrea; Pasteurella multocida; Pediococcus pentosaceus; Phormidium foveolarum; Phytoplasma sp. Plectonema boryanum; Prevotella ruminicola; Propionibacterium sp.; Proteus vulgaris; Pseudomonas sp.; Ralstonia sp.; Rhizobium sp.; Rhodococcus equi; Rhodothermus marinus; Rickettsia sp.; Riemerella anatipestifer; Ruminococcus flavefaciens; Salmonella sp.; Selenomonas ruminantium; Serratia entomophila; Shigella sp.; Sinorhizobium meliloti; Staphylococcus sp.; Streptococcus sp.; Streptomyces sp.; Synechococcus sp.; Synechocystis sp. PCC 6803; Thermotoga maritima; Treponema sp.; Ureaplasma urealyticum; Vibrio cholerae; Vibrio parahaemolyticus; Xylella fastidiosa; Yersinia sp.; Zymomonas mobilis, preferably Salmonella sp. or Escherichia coli or plants, preferably from yeasts such as from the genera Saccharomyces, Pichia, Candida, Hansenula, Torulopsis or Schizosaccharomyces, or even more preferred from plants such as Arabidopsis thaliana, maize, wheat, rye, oat, triticale, rice, barley, soybean, peanut, cotton, borage, safflower, linseed, primrose, rapeseed, canola and turnip rape, manihot, pepper, sunflower, tagetes, solanaceous plant such as potato, tobacco, eggplant and tomato, Vicia species, pea, alfalfa, bushy plants such as coffee, cacao, tea, Salix species, trees such as oil palm, coconut, perennial grass, such as ryegrass and fescue, and forage crops, such as alfalfa and clover and from spruce, pine or fir for example, more preferably from Saccharomyces cerevisiae or plants, preferably Brassica napus, Glycine max or Oryza sativa.

“Homologs” are defined herein as two nucleic acids or proteins that have similar, or “homologous”, nucleotide or amino acid sequences, respectively. Homologs include allelic variants, orthologs, paralogs, agonists and antagonists of SRP as defined hereafter. The term “homolog” further encompasses nucleic acid molecules that differ from one of the nucleotide sequences shown in sequences of FIG. 1a, 1b, 1c or 1d (and portions thereof) due to degeneracy of the genetic code and thus encode the same SRP as that encoded by the amino acid sequences shown in sequences of FIG. 1a, 1b, 1c or 1d. As used herein a “naturally occurring” SRP refers to a SRP amino acid sequence that occurs in nature.

The term “homology” means that the respective nucleic acid molecules or encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants. Structurally equivalents can, for example, be identified by testing the binding of said polypeptide to antibodies or computer based predictions. Structurally equivalent have the similar immunological characteristic, e.g. comprise similar epitopes.

Functional equivalents derived from one of the polypeptides as shown in FIG. 1a, 1b, 1c or 1d according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the polypeptides as shown in FIG. 1a, 1b, 1c or 1d according to the invention and are distinguished by essentially the same properties as the polypeptide as shown in FIG. 1a, 1b, 1c or 1d.

Functional equivalents derived from the nucleic acid sequence as shown in FIG. 1a, 1b, 1c or 1d according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the polypeptides as shown in FIG. 1a, 1b, 1c or 1d according to the invention and encode polypeptides having essentially the same properties as the polypeptide as shown in FIG. 1a, 1b, 1c or 1d.

“Essentially the same properties” of a functional equivalent is above all understood as meaning that the functional equivalent has above mentioned activity, e.g conferring an increase in the fine chemical amount while increasing the amount of protein, activity or function of said functional equivalent in an organism, e.g. a microorganism, a plant or plant or animal tissue, plant or animal cells or a part of the same.

By “hybridizing” it is meant that such nucleic acid molecules hybridize under conventional hybridization conditions, preferably under stringent conditions such as described by, e.g., Sambrook (Molecular Cloning; A Laboratory Manual, 2nd Edition, Cold Spring. Harbor Laboratory Press, Cold Spring Harbor, N.Y. (1989)) or in Current Protocols in Molecular Biology, John Wiley & Sons, N.Y. (1989), 6.3.1-6.3.6.

According to the invention, DNA as well as RNA molecules of the nucleic acid of the invention can be used as probes. Further, as template for the identification of functional homologues. Northern blot assays as well as Southern blot assays can be performed. The Northern blot assay advantageously provides further informations about the expressed gene product: e.g. expression pattern, occurrence of processing steps, like splicing and capping, etc. The Southern blot assay provides additional information about the chromosomal localization and organization of the gene encoding the nucleic acid molecule of the invention.

A preferred, nonlimiting example of stringent hydridization conditions are hybridizations in 6× sodium chloride/sodium citrate (=SSC) at approximately 45° C., followed by one or more wash steps in 0.2×SSC, 0.1% SDS at 50 to 65° C., for example at 50° C., 55° C. or 60° C. The skilled worker knows that these hybridization conditions differ as a function of the type of the nucleic acid and, for example when organic solvents are present, with regard to the temperature and concentration of the buffer. The temperature under “standard hybridization conditions” differs for example as a function of the type of the nucleic acid between 42° C. and 58° C., preferably between 45° C. and 50° C. in an aqueous buffer with a concentration of 0.1×0.5×, 1×, 2×, 3×, 4× or 5×SSC (pH 7.2). If organic solvent(s) is/are present in the abovementioned buffer, for example 50% formamide, the temperature under standard conditions is approximately 40° C., 42° C. or 45° C. The hybridization conditions for DNA:DNA hybrids are preferably for example 0.1×SSC and 20° C., 25° C., 30° C., 35° C., 40° C. or 45° C., preferably between 30° C. and 45° C. The hybridization conditions for DNA:RNA hybrids are preferably for example 0.1×SSC and 30° C., 35° C., 40° C., 45° C., 50° C. or 55° C., preferably between 45° C. and 55° C. The abovementioned hybridization temperatures are determined for example for a nucleic acid approximately 100 bp (=base pairs) in length and a G+C content of 50% in the absence of formamide. The skilled worker knows to determine the hybridization conditions required with the aid of textbooks, for example the ones mentioned above, or from the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford.

A further example of one such stringent hybridization condition is hybridization at 4×SSC at 65° C., followed by a washing in 0.1×SSC at 65° C. for one hour. Alternatively, an exemplary stringent hybridization condition is in 50% formamide, 4×SSC at 42° C. Further, the conditions during the wash step can be selected from the range of conditions delimited by low-stringency conditions (approximately 2×SSC at 50° C.) and high-stringency conditions (approximately 0.2×SSC at 50° C., preferably at 65° C.) (20×SSC: 0.3M sodium citrate, 3M NaCl, pH 7.0). In addition, the temperature during the wash step can be raised from low-stringency conditions at room temperature, approximately 22° C., to higher-stringency conditions at approximately 65° C. Both of the parameters salt concentration and temperature can be varied simultaneously, or else one of the two parameters can be kept constant while only the other is varied. Denaturants, for example formamide or SDS, may also be employed during the hybridization. In the presence of 50% formamide, hybridization is preferably effected at 42° C. Relevant factors like i) length of treatment, ii) salt conditions, iii) detergent conditions, iv) competitor DNAs, v) temperature and vi) probe selection can be combined case by case so that not all possibilities can be mentioned herein.

Thus, in a preferred embodiment, Northern blots are prehybridized with Rothi-Hybri-Quick buffer (Roth, Karlsruhe) at 68° C. for 2 h. Hybridization with radioactive labelled probe is done overnight at 68° C. Subsequent washing steps are performed at 68° C. with 1×SSC.

For Southern blot assays the membrane is prehybridized with Rothi-Hybri-Quick buffer (Roth, Karlsruhe) at 68° C. for 2 h. The hybridization with radioactive labelled probe is conducted over night at 68° C. Subsequently the hybridization buffer is discarded and the filter shortly washed using 2×SSC; 0,1% SDS. After discarding the washing buffer new 2×SSC; 0,1% SDS buffer is added and incubated at 68° C. for 15 minutes. This washing step is performed twice followed by an additional washing step using 1×SSC; 0,1% SDS at 68° C. for 10 min.

Some further examples of conditions for DNA hybridization (Southern blot assays) and wash step are shown hereinbelow:

(1) Hybridization conditions can be selected, for example, from the following conditions:

a) 4×SSC at 65° C.,
b) 6×SSC at 45° C.,
c) 6×SSC, 100 mg/ml denatured fragmented fish sperm DNA at 68° C.,
d) 6×SSC, 0.5% SDS, 100 mg/ml denatured salmon sperm DNA at 68° C.,
e) 6×SSC, 0.5% SDS, 100 mg/ml denatured fragmented salmon sperm DNA, 50% formamide at 42° C.,
f) 50% formamide, 4×SSC at 42° C.,
g) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C.,
h) 2× or 4×SSC at 50° C. (low-stringency condition), or
i) 30 to 40% formamide, 2× or 4×SSC at 42° C. (low-stringency condition).
   (2) Wash steps can be selected, for example, from the following conditions:
a) 0.015 M NaCl/0.0015 M sodium citrate/0.1% SDS at 50° C.
b) 0.1×SSC at 65° C.
c) 0.1×SSC, 0.5% SDS at 68° C.
d) 0.1×SSC, 0.5% SDS, 50% formamide at 42° C.
e) 0.2×SSC, 0.1% SDS at 42° C.
f) 2×SSC at 65° C. (low-stringency condition).

With regard to the invention described here, “transformed” means all those plants or parts thereof which have been brought about and/or modified by manipulation methods and in which either
a) one or more genes, preferably encoded by one or more nucleic acid sequence as depicted in sequences of FIG. 1a, 1b, 1c or 1d or a homolog thereof, or
b) a genetic regulatory element or elements, for example promoters, which are functionally linked e.g. to a nucleic acid sequence of sequences of FIG. 1a, 1b, 1 c or 1d or a homolog thereof, or
c) (a) and (b)

is/are not present in its/their natural genetic environment and/or has/have been modified by means of manipulation methods.

It is possible for the modification to be, by way of example, a substitution, addition, deletion, inversion or insertion of one or more nucleotides.

Manipulation in the present invention is also meant to encompass all changes in the plant cell, including induced or non-induced (spontaneous) mutagenesis, directed or non-directed genetic manipulation by conventional breeding or by modern genetic manipulation methods, e.g. reduction of gene expression by double-stranded RNA interference (dsRNAi), introduction of an antisense nucleic acid, a ribozyme, an antisense nucleic acid combined with a ribozyme, a nucleic acid encoding a co-suppressor, a nucleic acid encoding a dominant negative protein, DNA- or RNA- or protein-binding factors targeting said gene or -RNA or -proteins, RNA degradation inducing viral nucleic acids and expression systems, systems for inducing a homolog recombination of said genes, mutations in said genes or a combination of the above.

Additional modifications and manipulation methods will become apparent from the further description.

“Natural genetic environment” means the natural chromosomal locus in the organism of origin or the presence in a genomic library. In the case of a genomic library, the natural, genetic environment of the nucleic acid sequence is preferably at least partially still preserved. The environment flanks the nucleic acid sequence at least on one side and has a sequence length of at least 50 bp, preferably at least 500 bp, particularly preferably at least 1000 bp, very particularly preferably at least 5000 bp.

A plant or plant cell is considered “true breeding” for a particular attribute if it is genetically homozygous for that attribute to the extent that, when the true-breeding plant is self-pollinated, a significant amount of independent segregation of the attribute among the progeny is not observed.

As also used herein, the terms “nucleic acid” and “nucleic acid molecule” are intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. The nucleic acid molecule can be single-stranded or double-stranded, but preferably is double-stranded DNA.

An “isolated” nucleic acid molecule is one that is substantially separated from other nucleic acid molecules, which are present in the natural source of the nucleic acid. That means other nucleic acid molecules are present in an amount less than 5% based on weight of the amount of the desired nucleic acid, preferably less than 2% by weight, more preferably less than 1% by weight, most preferably less than 0.5% by weight. Preferably, an “isolated” nucleic acid is free of some of the sequences that naturally flank the nucleic acid (i.e., sequences located at the 5′ and 3′ ends of the nucleic acid) in the genomic DNA of the organism from which the nucleic acid is derived. For example, in various embodiments, the isolated gene encoding nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

A nucleic acid molecule of the present invention, e.g., a nucleic acid, molecule encoding a gene or a portion thereof or a homolog thereof which confers tolerance and/or resistance to environmental stress in plants, when inactivated or down-regulated, can be isolated using standard molecular biology techniques and the sequence information provided herein. For example, an Arabidopsis thaliana gene encoding cDNA can be isolated from an A. thaliana library using all or portion of one of sequences of the nucleic acid as shown in FIG. 1a, 1b, 1c or 1d. Moreover, a nucleic acid molecule encompassing all or a portion of one of the sequences of sequences of FIG. 1a, 1b, 1c or 1d can be isolated by the polymerase chain reaction using oligonucleotide primers designed based upon this sequence. For example, mRNA can be isolated from plant cells (e.g., by the guanidinium-thiocyanate extraction procedure of Chirgwin et al., 1979 Biochemistry 18:5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in FIG. 1a, 1b, 1c or 1d. A nucleic acid molecule of the invention can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to a gene encoding nucleotide sequence can be prepared by standard synthetic techniques, e.g., using an automated DNA synthesizer.

In a preferred embodiment, an isolated nucleic acid molecule of the invention comprises one of the nucleotide sequences shown in sequences of FIG. 1a, 1b, 1c or 1d or homologs thereof encoding a gene (i.e., the “coding region”), as well as 5′ untranslated sequences and 3′ untranslated sequences.

Moreover, the nucleic acid molecule of the invention can comprise only a portion of the coding region of one of sequences of FIG. 1a, 1b, 1c or 1d or homologs thereof, for example, a fragment which can be used as a probe or primer or a fragment encoding a biologically active portion of a gene.

Portions of genes or proteins encoded by said gene encoding nucleic acid molecules of the invention are preferably biologically active portions of genes or proteins described herein. As used herein, the term “biologically active portion of” a gene or protein encoded by said gene is intended to include a portion, e.g., a domain/motif, of the gene or protein that participates in stress tolerance and/or resistance response in a plant, which is preferably achieved by altering metabolic activity. To determine whether inactivation or down-regulation of a gene or protein encoded by said gene, or a biologically active portion thereof, results in increased stress tolerance in a plant which is preferably achieved by altering metabolic activity, a stress analysis of a plant comprising the protein may be performed for example by the above screening method. More specifically, nucleic acid fragments encoding biologically active portions of a gene or protein encoded by said gene can be prepared by isolating a portion of one of sequences of the nucleic acid as shown in FIG. 1a, 1b, 1c or 1d or homologs thereof expressing the encoded portion of the gene, protein or peptide (e.g., by recombinant expression in vitro) and assessing the activity of the encoded portion of the gene, protein or peptide.

Moreover, it is possible to identify conserved regions from various organisms by carrying out protein sequence alignments with the polypeptide used in the process of the invention, in particular with sequences of the polypeptide of the invention, from which conserved regions, and in turn, degenerate primers can be derived. Conserved region for the polypeptide of the invention are indicated in the alignments shown in the figures. Conserved regions are those, which show a very little variation in the amino acid in one particular position of several homologs from different origin.

Typically portions of a protein encompassed by the present invention include peptides comprising amino acid sequences derived from the amino acid sequence of the protein encoded by one of sequences of FIG. 1a, 1b, 1c or 1d, or the amino acid sequence of a protein homologous to the protein, which include fewer amino acids than the full length protein or a full length protein which is homologous to the protein, and exhibits at least some activity of the protein. Preferred portions according to the present invention (e.g., peptides or proteins which are, for example, 5, 10, 15, 20, 30, 35, 36, 37, 38, 39, 40, 50, 100 or more amino acids in length) comprise a domain or motif with at least some activity of the protein. Moreover, other biologically active portions in which other regions of the protein are deleted can be prepared by recombinant techniques and evaluated for one or more of the activities described herein. Preferably, the biologically active portions of the protein include one or more selected domains/motifs or portions thereof having biological activity.

In addition to fragments of the protein described herein, the present invention especially includes homologs and analogs of naturally occurring proteins and protein encoding nucleic acids in a plant.

“Homologs” are defined herein as two nucleic acids or proteins that have similar, or “homologous”, nucleotide or amino acid sequences, respectively. Homologs include allelic variants, orthologs, paralogs, agonists and antagonists of the protein as defined hereafter. The term “homolog” further encompasses nucleic acid molecules that differ from one of the nucleotide sequences shown in sequences of FIG. 1a, 1b, 1c or 1d (and portions thereof) due to degeneracy of the genetic code and thus encode the same protein as that encoded by the amino acid sequences. As used herein a “naturally occurring” refers to an amino acid sequence that occurs in nature.

In addition to fragments and fusion polypeptides of the invention described herein, the present invention includes homologs and analogs of naturally occurring proteins and protein encoding nucleic acids of the invention in a plant. “Homologs” are defined, herein as two nucleic acids or polypeptides that have similar, or substantially identical, nucleotide or amino acid sequences, respectively. Homologs include allelic variants, orthologs, paralogs, agonists and antagonists of SRPs as defined hereafter. The term “homolog” further encompasses nucleic acid molecules that differ from one of the nucleotide sequences shown in FIG. 1a, 1b, 1c or 1d (and portions thereof) due to degeneracy of the genetic code and thus encode the same SRP as that encoded by the nucleotide sequences shown in FIG. 1a, 1b, 1c or 1d. As used herein a “naturally occurring” protein refers to amino acid sequence that occurs in nature. Preferably, a naturally occurring protein whose reduction or deletion results in increased tolerance and/or resistance to an environmental stress comprises an amino acid sequence selected from the group consisting of ones shown in FIG. 1a, 1b, 1c or 1d.

An agonist of the protein whose reduction or deletion results in increased tolerance and/or resistance to an environmental stress can retain substantially the same, or a subset, of the biological activities of the said protein. An antagonist of the said protein can inhibit one or more of the activities of the naturally occurring form of the protein whose reduction or deletion results in increased tolerance and/or resistance to an environmental stress. For example, an antagonist can competitively bind to a downstream or upstream member of the cell membrane component metabolic cascade that includes said protein, or bind to the protein of the invention that mediates transport of compounds across such membranes, thereby preventing translocation from taking place.

Nucleic acid molecules corresponding to natural allelic variants and analogs, orthologs and paralogs of a protein of the invention cDNA can be isolated based on their identity to the Arabidopsis thaliana, Saccharomyces cerevisiae, E. coli, Brassica napus, Glycine max, or Oryza sativa protein nucleic acids described herein using said proteins cDNAs, or a portion thereof, as a hybridization probe according to standard hybridization techniques under stringent hybridization conditions. In an alternative embodiment, homologs of the protein whose reduction or deletion results in increased tolerance and/or resistance to an environmental stress can be identified by screening combinatorial libraries of mutants, e.g., truncation mutants, of said protein for their agonist or antagonist activity. In one embodiment, a variegated library of protein whose reduction or deletion results in increased tolerance and/or resistance to an environmental stress variants is generated by combinatorial mutagenesis at the nucleic acid level and is encoded by a variegated gene library. A variegated library of SRP variants can be produced by, for example, enzymatically ligating a mixture of synthetic oligonucleotides into gene sequences such that a degenerate set of potential SRP sequences is expressible as individual polypeptides, or alternatively, as a set of larger fusion polypeptides (e.g., for phage display) containing the set of protein whose reduction or deletion results in increased tolerance and/or resistance to an environmental stress sequences therein. There are a variety of methods that can be used to produce libraries of potential protein homologs from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential protein of the invention sequences. Methods for synthesizing degenerate oligonucleotides are known in the art. See, e.g., Narang, S. A., 1983, Tetrahedron 39:3; Itakura et al., 1984, Annu. Rev. Biochem. 53:323; Itakura et al., 1984, Science 198:1056; Ike et al., 1983, Nucleic Acid Res. 11:477.

In addition, libraries of fragments of the protein of the invention coding regions can be used to generate a variegated population of protein fragments for screening and subsequent selection of homologs of a said proteins. In one embodiment, a library of coding sequence fragments can be generated by treating a double stranded PCR fragment of a protein of the invention coding sequence with a nuclease under conditions wherein nicking occurs only about once per molecule, denaturing the double stranded DNA, renaturing the DNA to form double stranded DNA, which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal, and internal fragments of various sizes of the protein whose reduction or deletion results in increased tolerance and/or resistance to an environmental stress.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SRP homologs. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify SRP homologs (Arkin and Yourvan, 1992, PNAS 89:7811-7815; Delgrave et al., 1993, Polypeptide Engineering 6(3):327-331). In another embodiment, cell based assays can be exploited to analyze a variegated protein (of the invention) library, using methods well known in the art. The present invention further provides a method of identifying a novel protein whose reduction or deletion results in increased tolerance and/or resistance to an environmental stress, comprising (a) raising a specific antibody response to said protein, or a fragment thereof, as described herein; (b) screening putative SRP material with the antibody, wherein specific binding of the antibody to the material indicates the presence of a potentially novel protein whose reduction or deletion results in increased tolerance and/or resistance to an environmental stress; and (c) analyzing the bound material in comparison to known proteins, to determine its novelty.

As stated above, the present invention includes protein whose reduction or deletion results in increased tolerance and/or resistance to an environmental stress and homologs thereof. To determine the percent sequence identity of two amino acid sequences (e.g., one of the sequences of FIG. 1a, 1b, 1c or 1d, and a mutant form thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence (e.g., one of the sequences of FIG. 1a, 1b, 1c or 1d) is occupied by the same amino acid residue as the corresponding position in the other sequence (e.g., a mutant form of the sequence selected from the polypeptide of FIG. 1a, 1b, 1c or 1d), then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions×100). Preferably, the isolated amino acid homologs included in the present invention are at least about 50-60%, preferably at least about 60-70%, and more preferably at least about 70-75%, 75-80%, 80-85%, 85-90% or 90-95%, and most preferably at least about 96%, 97%, 98%, 99% or more identical to an entire amino acid sequence shown in FIG. 1a, 1b, 1c or 1d. In yet another embodiment, the isolated amino acid homologs included in the present invention are at least about 50-60%, preferably at least about 60-70%, and more preferably at least about 70-75%, 75-80%, 80-85%, 85-90% or 90-95%, and most preferably at least about 96%, 97%, 98%, 99% or more identical to an entire amino acid sequence encoded by a nucleic acid sequence shown in FIG. 1a, 1b, 1c or 1d. In other embodiments, the amino acid homologs of the proteins of the invention have sequence identity over at least 15 contiguous amino acid residues, more preferably at least 25 contiguous amino acid residues, and most preferably at least 35 contiguous amino acid residues of a polypeptide of FIG. 1a, 1b, 1c or 1d.

In another preferred embodiment, an isolated nucleic acid homolog of the invention comprises a nucleotide sequence which is at least about 50-60%, preferably at least about 60-70%, more preferably at least about 70-75%, 75-80%, 80-85%, 85-90% or 90-95%, and even more preferably at least about 95%, 96%, 97%, 98%, 99% or more identical to a nucleotide sequence shown in FIG. 1a, 1b, 1c or 1d, or to a portion comprising at least 20, 30, 40, 50, 60 consecutive nucleotides thereof. The preferable length of sequence comparison for nucleic acids is at least 75 nucleotides, more preferably at least 100 nucleotides and most preferably the entire length of the coding region.

It is further preferred that the isolated nucleic acid homolog of the invention encodes a SRP, or portion thereof, that is at least 85% identical to an amino acid sequence of FIG. 1a, 1b, 1 c or 1d and that functions as a modulator of an environmental stress response in a plant. In a more preferred embodiment, overexpression of the nucleic acid homolog in a plant increases the tolerance of the plant to an environmental stress.

For the purposes of the invention, the percent sequence identity between two nucleic acid or polypeptide sequences may be determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

In another aspect, the invention provides an isolated nucleic acid comprising a polynucleotide that hybridizes to the polynucleotide of FIG. 1a, 1b, 1c or 1d under stringent conditions. More particularly, an isolated nucleic acid molecule of the invention is at least 15 nucleotides in length and hybridizes under stringent conditions to the nucleic acid molecule comprising a nucleotide sequence of FIG. 1a, 1b, 1c or 1d. In other embodiments, the nucleic acid is at least 30, 50, 100, 250 or more nucleotides in length. Preferably, an isolated nucleic acid homolog of the invention comprises a nucleotide sequence which hybridizes under highly stringent conditions to the nucleotide sequence shown in FIG. 1a, 1b, 1c or 1d, and functions as a modulator of stress tolerance in a plant. In a further preferred embodiment, overexpression of the isolated nucleic acid homolog in a plant increases a plant's tolerance to an environmental stress.

As used herein with regard to hybridization for DNA to DNA blot, the term “stringent conditions” refers in one embodiment to hybridization overnight at 60° C. in 10× Denharts solution, 6×SSC, 0.5% SDS and 100 μg/ml denatured salmon sperm DNA. Blots are washed sequentially at 62° C. for 30 minutes each time in 3×SSC/0.1% SDS, followed by 1×SSC/0.1% SDS and finally 0.1×SSC/0.1% SDS. As also used herein, “highly stringent conditions” refers to hybridization overnight at 65° C. in 10× Denharts solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS and finally 0.1×SSC/0.1% SDS. Methods for nucleic acid hybridizations are described in Meinkoth and Wahl, 1984, Anal. Biochem. 138:267-284; Ausubel et al. eds, 1995, Current Protocols in Molecular Biology, Chapter 2, Greene Publishing and Wiley-Interscience, New York; and Tijssen, 1993, Laboratory Techniques in Biochemistry and Molecular Biology: Hybridization with Nucleic Acid Probes, Part I, Chapter 2, Elsevier, New York. Preferably, an isolated nucleic acid molecule of the invention that hybridizes under stringent or highly stringent conditions to a sequence of FIG. 1a, 1b, 1c or 1d corresponds to a naturally occurring nucleic acid molecule. As used herein, a “naturally occurring” nucleic acid molecule refers to an RNA or DNA molecule having a nucleotide sequence that occurs in nature (e.g., encodes a natural polypeptide). In one embodiment, the nucleic acid encodes a naturally occurring Arabidopsis thaliana, Brassica napus, Glycine max, or Oryza sativa protein whose reduction or deletion results in increased tolerance and/or resistance to an environmental stress.

Using the above-described methods, and others known to those of skill in the art, one of ordinary skill in the art can isolate homologs of the protein whose reduction or deletion results in increased tolerance and/or resistance to an environmental stress comprising amino acid sequences shown in FIG. 1a, 1b, 1c or 1d. One subset of these homologs are allelic variants. As used herein, the term “allelic variant” refers to a nucleotide sequence containing polymorphisms that lead to changes in the amino acid sequences of said protein and that exist within a natural population (e.g., a plant species or variety). Such natural allelic variations can typically result in 1-5% variance in a nucleic acid of the invention. Allelic variants can be identified by sequencing the nucleic acid sequence of interest in a number of different plants, which can be readily carried out by using hybridization probes to identify the same SRP genetic locus in those plants. Any and all such nucleic acid variations and resulting amino acid polymorphisms or variations in a protein whose reduction or deletion results in increased tolerance and/or resistance to an environmental stress that are the result of natural allelic variation and that do not alter the functional activity of said protein, are intended to be within the scope of the invention.

An isolated nucleic acid molecule encoding a protein whose reduction or deletion results in increased tolerance and/or resistance to an environmental stress having sequence identity with a polypeptide sequence of FIG. 1a, 1b, 1c or 1d can be created by introducing one or more nucleotide substitutions, additions or deletions into a nucleotide sequence of FIG. 1a, 1b, 1c or 1d, respectively, such that one or more amino acid substitutions, additions, or deletions are introduced into the encoded polypeptide. Mutations can be introduced into one of the sequences of FIG. 1a, 1b, 1c or 1d by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

To knock the mutation is carried out preferably at essential positions!

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein of the invention is preferably replaced with another amino acid residue from the same side chain family. Alternatively, in another embodiment, mutations can be introduced randomly along all or part of a protein of the invention coding sequence, such as by saturation mutagenesis, and the resultant mutants can be screened for a SRP activity described herein to identify mutants that retain protein activity. Following mutagenesis of one of the sequences, of FIG. 1a, 1b, 1c or 1d, the encoded polypeptide can be expressed recombinantly and the activity of the polypeptide can be determined by analyzing the stress tolerance of a plant expressing the polypeptide as described herein.

In addition to the nucleic acid molecules encoding the protein whose reduction or deletion results in increased tolerance and/or resistance to an environmental stress described above, another aspect of the invention pertains to isolated nucleic acid molecules that are antisense thereto. Antisense polynucleotides are thought to inhibit gene expression of a target polynucleotide by specifically binding the target polynucleotide and interfering with transcription, splicing, transport, translation, and/or stability of the target polynucleotide. Methods are described in the prior art for targeting the antisense polynucleotide to the chromosomal DNA, to a primary RNA transcript, or to a processed mRNA. Preferably, the target regions include splice sites, translation initiation codons, translation termination codons, and other sequences within the open reading frame.

The term “antisense,” for the purposes of the invention, refers to a nucleic acid comprising a polynucleotide that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. The term “antisense nucleic acid” includes single stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity with the polypeptide of FIG. 1a, 1b, 1c or 1d.

The antisense nucleic acid can be complementary to an entire SRP coding strand, or to only a portion thereof. In one embodiment, an antisense nucleic acid molecule is antisense to a “coding region” of the coding strand of a nucleotide sequence encoding a SRP. The term “coding region” refers to the region of the nucleotide sequence comprising codons that are translated into amino acid residues. In another embodiment, the antisense nucleic acid molecule is antisense to a “noncoding region” of the coding strand of a nucleotide sequence encoding a SRP. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of SRP mRNA, but more preferably is an oligonucleotide which is antisense to only a portion of the coding or noncoding region of SRP mRNA. For example, the antisense oligonucleotide can be complementary to the region surrounding the translation start site of PKSRP mRNA. An antisense oligonucleotide can be, for example, about 5, 10, 15, 20, 25, 30, 35, 40, 45 or 50 nucleotides in length. Typically, the antisense molecules of the present invention comprise an RNA having 60-100% sequence identity with at least 14 consecutive nucleotides of FIG. 1a, 1b, 1c or 1d. Preferably, the sequence identity will be at least 70%, more preferably at least 75%, 80%, 85%, 90%, 95%, 98% and most preferably 99%.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine. N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The antisense nucleic acid molecules of the invention are typically administered to a cell or generated in situ such that they hybridize with or bind to cellular mRNA and/or genomic DNA encoding a SRP to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen, The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic (including plant) promoter are preferred.

As an alternative to antisense polynucleotides, ribozymes, sense polynucleotides, or double stranded RNA (dsRNA) can be used to reduce expression of a SRP polypeptide. By “ribozyme” is meant a catalytic RNA-based enzyme with ribonuclease activity which is capable of cleaving a single-stranded nucleic acid, such as an mRNA, to which it has a complementary region. Ribozymes (e.g., hammerhead ribozymes described in Haselhoff and Gerlach, 1988, Nature 334:585-591) can be used to catalytically cleave proteins of the invention mRNA transcripts to thereby inhibit translation of SR P mRNA. A ribozyme having specificity for a nucleic acid of the invention can be designed based upon the nucleotide sequence of a cDNA, as disclosed herein (i.e., sequences as shown in FIG. 1a, 1b, 1c or 1d) or on the basis of a heterologous sequence to be isolated according to methods taught in this invention. For example, a derivative of a Tetrahymena L-19 IVS RNA can be constructed in which the nucleotide sequence of the active site is complementary to the nucleotide sequence to be cleaved in a SRP-encoding mRNA. See, e.g., U.S. Pat. Nos. 4,987,071 and 5,116,742 to Cech et al. Alternatively, SRP mRNA can be used to select a catalytic RNA having a specific ribonuclease activity from a pool of RNA molecules. See, e.g., Bartel, D. and Szostak, J. W., 1993, Science 261:1411-1418. In preferred embodiments, the ribozyme will contain a portion having at least 7, 8, 9, 10, 12, 14, 16, 18 or 20 nucleotides, and more preferably 7 or 8 nucleotides, that have 100% complementarity to a portion of the target RNA. Methods for making ribozymes are known to those skilled in the art. See, e.g., U.S. Pat. Nos. 6,025,167; 5,773,260; and 5,496,698.

The term “dsRNA,” as used herein, refers to RNA hybrids comprising two strands of RNA. The dsRNAs can be linear or circular in structure. In a preferred embodiment, dsRNA is specific for a polynucleotide encoding either the polypeptide of FIG. 1a, 1b, 1c or 1d or a polypeptide having at least 70% sequence identity with FIG. 1a, 1b, 1c or 1d. The hybridizing RNAs may be substantially or completely complementary. By “substantially complementary,” is meant that when the two hybridizing RNAs are optimally aligned using the BLAST program as described above, the hybridizing portions are at least 95% complementary. Preferably, the dsRNA will be at least 100 base pairs in length. Typically, the hybridizing RNAs will be of identical length with no over hanging 5′ or 3′ ends and no gaps. However, dsRNAs having 5′ or 3′ overhangs of up to 100 nucleotides may be used in the methods of the invention.

The dsRNA may comprise ribonucleotides or ribonucleotide analogs, such as 2′-O-methyl ribosyl residues, or combinations thereof. See, e.g., U.S. Pat. Nos. 4,130,641 and 4,024,222. A dsRNA polyriboinosinic acid:polyribocytidylic acid is described in U.S. Pat. No. 4,283,393. Methods for making and using dsRNA are known in the art. One method comprises the simultaneous transcription of two complementary DNA strands, either in vivo, or in a single in vitro reaction mixture. See, e.g., U.S. Pat. No. 5,795,715. In one embodiment, dsRNA can be introduced into a plant or plant cell directly by standard transformation procedures. Alternatively, dsRNA can be expressed in a plant cell by transcribing two complementary RNAS.

Other methods for the inhibition of endogenous gene expression, such as triple helix formation (Moser et al., 1987, Science 238:645-650 and Cooney et al., 1988, Science 241:456-459) and cosuppression (Napoli et al., 1990, The Plant Cell 2:279-289) are known in the art. Partial and full-length cDNAs have been used for the cosuppression of endogenous plant genes. See, e.g., U.S. Pat. Nos. 4,801,340, 5,034,323, 5,231,020, and 5,283,184; Van der Kroll et al., 1990, The Plant Cell 2:291-299; Smith et al., 1990, Mol. Gen. Genetics 224:477-481 and Napoli et al., 1990, The Plant Cell 2:279-289.

For sense suppression, it is believed that introduction of a sense polynucleotide blocks transcription of the corresponding target gene. The sense polynucleotide will have at least 65% sequence identity with the target plant gene or RNA. Preferably, the percent identity is at least 80%, 90%, 95% or more. The introduced sense polynucleotide need not be full length relative to the target gene or transcript. Preferably, the sense polynucleotide will have at least 65% sequence identity with at least 100 consecutive nucleotides of FIG. 1a, 1b, 1c or 1d. The regions of identity can comprise introns and and/or exons and untranslated regions. The introduced sense polynucleotide may be present in the plant cell transiently, or may be stably integrated into a plant chromosome or extrachromosomal replicon.

Moreover, nucleic acid molecules encoding proteins from the same or other species such as protein analogs, orthologs and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species that have evolved from a common ancestral gene by speciation. Normally, orthologs encode proteins having the same or similar functions. As also used herein, the term “paralogs” refers to two nucleic acids that are related by duplication within a genome. Paralogs usually have different functions, but these functions may be related (Tatusov, R. L. et al. 1997 Science 278(5338):631-637). Analogs, orthologs and paralogs of naturally occurring proteins can differ from the naturally occurring proteins by post-translational modifications, by amino acid sequence differences, or by both. Post-translational modifications include in vivo and in vitro chemical derivatisation of polypeptides, e.g., acetylation, carboxylation, phosphorylation, or glycosylation, and such modifications may occur during polypeptide synthesis or processing or following treatment with isolated modifying enzymes. In particular, orthologs of the invention will generally exhibit at least 30%, more preferably 50%, and most preferably 90%, 91%, 92%, 93%, 94%, 95%, 96%, 97%, 98% or even 99% identity or homology with all or part of a naturally occurring protein amino acid sequence and will exhibit a function similar to a protein.

Such homologs, analogs, ortholdgs and paralogs will be referred to in general as homologs or being homologous throughout the present application.

Homologs of the sequences given in FIG. 1a, 1b, 1c or 1d are furthermore to be understood as meaning, for example, homologs, analogs, orthologs and paralogs which have at least 30% homology (=identity) at the derived amino acid level, preferably at least 50%, 60%, 70% or 80% homology, especially preferably at least 85% homology, very especially preferably 90%, 91%, 92%, 93%, 94%, homology, most preferably 95%, 96%, 97%, 98% or 99% homology. The homology (=identity) was calculated over the entire amino acid range. The program used was PileUp (J. Mol. Evolution., 25 (1987), 351-360, Higgens et al., CABIOS, 5 1989: 151-153) or the program. Gap and BestFit [Needleman and Wunsch (J. Mol. Biol. 48; 443-453 (1970) and Smith and Waterman respectively (Adv. Appl. Math. 2; 482-489 (1981)] which are part of the GCG software package [Genetics Computer Group, 575 Science Drive, Madison, Wis., USA 53711 (1991)]. The above mentioned percentages of sequence homology are calculated with the program BestFit or Gap, preferably Gap, over the total sequence length with the following parameters used: Gap Weight: 8, Length Weight: 2.

Furthermore the invention provides a method of producing a transformed plant, wherein inactivation or down-regulation of a gene in the transformed plant results in increased tolerance and/or resistance to environmental stress, which is preferably achieved by altering metabolic activity, as compared to a corresponding non-transformed wild type plant, comprising
(a) transforming a plant cell by inactivation or down-regulation of one or more genes, preferably encoded by one or more nucleic acids selected from a group consisting of th nucleic acids as shown in FIG. 1a, 1b, 1c or 1d and/or homologs thereof and
(b) generating from the plant cell a transformed plant with an increased tolerance and/or resistance to environmental stress as compared to a corresponding wild type plant.

The invention also incorporates a method of inducing increased tolerance and/or resistance to environmental stress as compared to a corresponding non-transformed wild type plant in said plant cell or said plant by altering metabolic activity, preferably of the above metabolites by inactivation or down-regulation of one or more genes encoded by one or more nucleic acids selected from a group consisting of the nucleic acids as shown in FIG. 1a, 1b, 1c or 1d and/or homologs thereof.

Preferably the nucleic acid is at least about 30%, especially at least 50% homologous to said sequence (see above). It is also possible that the homolog sequence stems form a plant selected from the group comprised of maize, wheat; rye, oat, triticale, rice, barley, soybean, peanut, cotton, rapeseed, canola, manihot, pepper, sunflower, flax, borage, safflower, linseed, primrose, rapeseed, turnip rape, tagetes, solanaceous plants, potato, tobacco, eggplant, tomato, Vicia species, pea, alfalfa, coffee, cacao, tea, Salix species, oil palm, coconut, perennial grass, forage crops and Arabidopsis thaliana, Brassica napus, Glycine max, or Oryza sativa.

Inactivation or down-regulation of said gene or genes may be achieved by all methods known to one skilled in the art, preferably by double-stranded RNA interference (dsRNAi), introduction of an antisense nucleic acid, a ribozyme, an antisense nucleic acid combined with a ribozyme, a nucleic acid encoding a co-suppressor, a nucleic acid encoding a dominant negative protein, DNA- or protein-binding factors targeting said gene or -RNA or -proteins, RNA degradation inducing viral nucleic acids and expression systems, systems for inducing a homolog recombination of said genes, mutations in said genes or a combination of the above.

The nucleic acid sequences of the invention or their homologs are isolated nucleic acid sequences which encode polypeptides. These nucleic acids or the polypeptides encoded by them and their biological and enzymatic activity are inactivated or downregulated in the method according to the invention which leads to increased resistance and/or tolerance to environmental stress, which is preferably achieved by altering metabolic activity.

In this context, inactivation means that the enzymatic or biological activity of the polypeptides encoded is no longer detectable in the organism or in the cell such as, for example, within the plant or plant cell. For the purposes of the invention, downregulation (=reduction) means that the enzymatic or biological activity of the polypeptides encoded is partly or essentially completely reduced in comparison with the activity of the untreated organism. This can be achieved by different cell-biological mechanisms. In this context, the activity can be downregulated in the entire organism or, in the case of multi-celled organisms, in individual parts of the organism, in the case of plants for example in tissues such as the seed, the leaf, the root or other parts. In this context, the enzymatic activity or biological activity is reduced by at least 10%, advantageously at least 20%, preferably at least 30%, especially preferably at least 40%, 50% or 60%, very especially preferably at least 70%, 80%, 90% or 95%, 99% or even 100% in comparison with the untreated organism. A particularly advantageous embodiment is the inactivation of the nucleic acids or of the polypeptides encoded by them.

Various strategies for reducing the quantity (expression), the activity or the function of proteins encoded by the nucleic acids according to the invention are encompassed in accordance with the invention. The skilled worker writ recognize that a series of different methods are available for influencing the quantity of a protein, the activity or the function in the desired manner.

A reduction in the activity or the function is preferably achieved by a reduced expression of a gene encoding an endogenous protein.

A reduction in the protein quantity, the activity or function can be achieved using the following methods:
a) introduction of a double-stranded RNA nucleic acid sequence (dsRNA) or of an expression cassette, or more than one expression cassette, ensuring the expression of the latter;
b) introduction of an antisense nucleic acid sequence or of an expression cassette ensuring the expression of the latter. Encompassed are those methods in which the antisense nucleic acid sequence is directed against a gene (i.e. genomic DNA sequences) or a gene transcript (i.e. RNA, sequences). Also encompassed are α-anomeric nucleic acid sequences.
c) introduction of an antisense nucleic acid sequence in combination with a ribozyme or of an expression cassette ensuring the expression of the former
d) introduction of sense nucleic acid sequences for inducing cosuppression or of an expression cassette ensuring the expression of the former
e) introduction of a nucleic acid sequence encoding dominant-negative protein or of an expression cassette ensuring the expression of the latter
f) introduction of DNA-, RNA- or protein-binding factors against genes, RNA's or proteins or of an expression cassette ensuring the expression of the latter
g) introduction of viral nucleic acid sequences and expression constructs which bring about the degradation of RNA, or of an expression cassette ensuring the expression of the former
h) introduction of constructs for inducing homologous recombination on endogenous genes, for example for generating knockout mutants.
i) introduction of mutations into endogenous genes for generating a loss of function (e.g. generation of stop codons, reading-frame shifts and the like)

Each of these methods may bring about a reduction in the expression, the activity or the function for the purposes of the invention. A combined use is also feasible. Further methods are known to the skilled worker and may encompass hindering or preventing processing of the protein, transport of the protein or its mRNA, inhibition of ribosomal attachment, inhibition of RNA splicing, induction of an enzyme which degrades RNA and/or inhibition of translational elongation or termination.

The term “protein quantity” refers to the amount of a polypeptide in an organism, a tissue, a cell or cell compartment. The term “reduction” of the protein quantity refers to the quantitative reduction of the amount of a protein in an organism, a tissue, a cell or a cell compartment—for example by one of the methods described herein below—in comparison with the wild type of the same genus and species to which this method has not been applied under otherwise identical conditions (such as, for example, culture conditions, age of the plants and the like). In this context, a reduction of at least 10%, advantageously of at least 20%, preferably at least 30%, especially preferably of at least 40%, 50% or 60%, very especially preferably of at least 70%, 80%, 90% or 95%, 99% or even 100% in comparison with the untreated organism is advantageous. An especially advantageous embodiment is the inactivation of the nucleic acids, or of the polypeptides encoded by them.

The term “activity” preferably refers to the activity of a polypeptide in an organism, a tissue, a cell or a cell compartment. The term “reduction” in the activity refers to the reduction in the overall activity of a protein in an organism, a tissue, a cell or a cell compartment—for example by one of the methods described herein below—in comparison with the wild type of the same genus and species, to which this method has not been applied, under otherwise identical conditions (such as, for example, culture conditions, age of the plants and the like). In this context, a reduction in activity of at least 10%, advantageously of at least 20%, preferably at least 30%, especially preferably of at least 40%, 50% or 60%, very especially preferably of at least 70%, 80%, 90% or 95%, 99% or even 100% in comparison with the untreated organism is advantageous. A particularly advantageous embodiment is the inactivation of the nucleic acids or of the polypeptides encoded by them.

The term “function” preferably refers to the enzymatic or regulatory function of a peptide in an organism, a tissue, a cell or a cell compartment. Suitable substrates are low-molecular-weight compounds and also the protein interaction partners of a protein. The term “reduction” of the function refers, for example, to the quantitative reduction in binding capacity or binding strength of a protein for at least one substrate in an organism, a tissue, a cell or a cell compartment—for example by one of the methods described herein below—in comparison with the wild type of the same genus and species to which this method has not been applied, under otherwise identical conditions (such as, for example, culture conditions, age of the plants and the like). Reduction is also understood as meaning the modification of the substrate specificity as can be expressed for example, by the kcat/Km value. In, this context, a reduction of the function of at least 10%, advantageously of at least 20%, preferably at least 30%, especially preferably of at least 40%, 50% or 60%, very especially preferably of at least 70%, 80%, 90010 or 95%, 99% or even 100% in comparison with the untreated organism is advantageous. A particularly advantageous embodiment is the inactivation of the function. Binding partners for the protein can be identified in the manner with which the skilled worker is familiar, for example by the yeast 2-hybrid system.

What follows is a brief description of the individual preferred methods:

A) Introduction of a Double-Stranded RNA Nucleic Acid Sequence dsRNA)

The method of regulating genes by means of double-stranded RNA (“double-stranded RNA interference”; dsRNAi) has been described extensively for animal and plant organisms (for example Matzke M A et al. (2000) Plant Mol. Biol. 43: 401-415; Fire A. et al. (1998) Nature 391: 806-811; WO 99/32619; WO 99/53050; WO 00/68374; WO 00/44914; WO 00/44895; WO 00/49035; WO 00/63364). The techniques and methods described in the above references are expressly referred to. Efficient gene suppression can also be observed in the case of transient expression or following transient transformation, for example as the consequence of a biolistic transformation (Schweizer P et al. (2000) Plant J 2000 24: 895-903). dsRNAi methods are based on the phenomenon that the simultaneous introduction of complementary strand and counterstrand of a gene transcript brings about highly effective suppression of the expression of the gene in question. The resulting phenotype is very similar to that of an analogous knock-out mutant (Waterhouse P M et al. (1998) Proc. Natl. Acad. Sci. USA 95: 13959-64).

Tuschl et al. [Gens Dev., 1999, 13 (24): 3191-3197] was able to show that the efficiency of the RNAi method is a function of the length of the duplex, the length of the 3′-end overhangs, and the sequence in these overhangs. Based on the work of Tuschl et al. and assuming that the underlining principles are conserved between different species the following guidelines can be given to the skilled worker:
to achieve good results the 5′ and 3′ untranslated regions of the used nucleic acid sequence and regions close to the start codon should be in general avoided as this regions are richer in regulatory protein binding sites and interactions between RNAi sequences and such regulatory proteins might lead to undesired interactions;
in plants the 5′ and 3′ untranslated regions of the used nucleic acid sequence and regions close to the start codon preferably 50 to 100 nt upstream of the start codon give good results and therefore should not be avoided;
preferably a region of the used mRNA is selected, which is 50 to 100 nt (=nucleotides or bases) downstream of the AUG start codon;
only dsRNA (=double-stranded RNA) sequences from exons are useful for the method, as sequences from introns have no effect;
the G/C content in this region should be greater than 30% and less than 70% ideally around 50%;
a possible secondary structure of the target mRNA is less important for the effect of the RNAi method.

The dsRNAi method has proved to be particularly effective and advantageous for reducing the expression of the nucleic acid sequences of sequences with odd numbers of SEQ ID No.'s 1-89 and/or homologs thereof. As described inter alia in WO 99/32619, dsRNAi approaches are clearly superior to traditional antisense approaches.

The invention therefore furthermore relates to double-stranded RNA molecules (dsRNA molecules) which, when introduced into an organism, advantageously into a plant (or a cell, tissue, organ or seed derived therefrom), bring about the reduction in the expression of the nucleic acid sequences of FIG. 1a, 1b, 1c or 1d and/or homologs thereof. In a double-stranded RNA molecule for reducing the expression of an protein encoded by a nucleic acid sequence of one of sequences of FIG. 1a, 1b, 1c or 1d and/or homologs thereof,
i) one of the two RNA strands is essentially identical to at least part of a nucleic acid sequence, and
ii) the respective other RNA strand is essentially identical to at least part of the complementary strand of a nucleic acid sequence.

The term “essentially identical” refers to the fact that the dsRNA sequence may also include insertions, deletions and individual point mutations in comparison to the target sequence while still bringing about an effective reduction in expression. Preferably, the homology as defined above amounts to at least 75%, preferably at least 80%, very especially preferably at least 90%, most preferably 100%, between the “sense” strand of an inhibitory dsRNA and a part-segment of a nucleic acid sequence of the invention (or between the “antisense” strand and the complementary strand of a nucleic acid sequence, respectively). The part-segment amounts to at least 10 bases, preferably at least 25 bases, especially preferably at least 50 bases, very especially preferably at least 100 bases, most preferably at least 200 bases or at least 300 bases in length. As an alternative, an “essentially identical” dsRNA may also be defined as a nucleic acid sequence which is capable of hybridizing with part of a gene transcript (for example in 400 mM NaCl, 40 mM PIPES pH 6.4, 1 mM EDTA at 50° C. or 70° C. for 12 to 16 h).

The dsRNA may consist of one or more strands of polymerized ribonucleotides. Modification of both the sugar-phosphate backbone and of the nucleosides may furthermore be present. For example, the phosphodiester bonds of the natural RNA can be modified in such a way that they encompass at least one nitrogen or sulfur hetero atom. Bases may undergo modification in such a way that the activity of, for example, adenosine deaminase is restricted. These and other modifications are described herein below in the methods for stabilizing antisense RNA.

The dsRNA can be prepared enzymatically; it may also be synthesized chemically, either in full or in part.

Short dsRNA up to 30bp, which effectively mediate RNA interference, can be for example efficiently generated by partial digestion of long dsRNA templates using E. coli ribonuclease III (RNase III). (Yang, D., et al. (2002) Proc. Natl. Acad. Sci. USA 99, 9942.)

The double-stranded structure can be formed starting from a single, self-complementary strand or starting from two complementary strands. In a single, self-complementary strand, “sense” and “antisense” sequence can be linked by a linking sequence (“linker”) and form for example a hairpin structure. Preferably, the linking sequence may take the form of an intron, which is spliced out following dsRNA synthesis. The nucleic acid sequence encoding a dsRNA may contain further elements such as, for example, transcription termination signals or polyadenylation signals. If the two strands of the dsRNA are to be combined in a cell or an organism advantageously in a plant, this can be brought about in a variety of ways:

a) transformation of the cell or of the organism, advantageously of a plant, with a vector encompassing the two expression cassettes,

b) cotransformation of the cell or of the organism, advantageously of a plant, with two vectors, one of which encompasses the expression cassettes with the “sense” strand while the other encompasses the expression cassettes with the “antisense” strand.

c) hybridization of two organisms, advantageously of plants, each of which has been transformed with one vector, one of which encompasses the expression cassettes with the “sense” strand while the other encompasses the expression cassettes with the “antisense” strand.

d) supertransformation of the cell or of the organism, advantageously of a plant, with a vector encompassing the expression cassettes with the “sense” strand, after the cell or the organism had already been transformed with a vector encompassing the expression cassettes with the “antisense” strand;

e) introduction of a construct comprising two promoters that lead to transcription of the desired sequence from both directions; and/or

f) infecting of the cell or of the organism with, advantageously of a plant, with an engeniered virus, which is able to produce the disered dsRNA molecule.

Formation of the RNA duplex can be initiated either outside the cell or within the cell.

If the dsRNA is synthesized outside the target cell or organism it can be introduced into the organism or a cell of the organism by injection, microinjection, electroporation, high velocity particles, by laser beam or mediated by chemical compounds (DEAE-dextran, calciumphosphate, liposomes) or in case of animals it is also possible to feed bacteria such as E. coli strains engineered to express-double-stranded RNAi to the animals.

As shown in WO 99/53050, the dsRNA may also encompass a hairpin structure, by linking the “sense” and “antisense” strands by a “linker” (for example an intron). The self-complementary dsRNA structures are preferred since they merely require the expression of a construct and always encompass the complementary strands in an equimolar ratio.

As shown in WO 99/53050, the dsRNA may also encompass a hairpin structure, by linking the “sense” and “antisense” strands by a “linker” (for example an intron). The self-complementary dsRNA structures are preferred since they merely require the expression of a construct and always encompass the complementary strands in an equimolar ratio.

The expression cassettes encoding the “antisense” or the “sense” strand of the dsRNA or the self-complementary strand of the dsRNA are preferably inserted into a vector and stably inserted into the genome of a plant, using the methods described herein below (for example using selection markers), in order to ensure permanent expression of the dsRNA.

The dsRNA can be introduced using an amount which makes possible at least one copy per cell. A larger amount (for example at least 5, 10, 100, 500 or 1000 copies per cell) may bring about more efficient reduction.

As has already been described, 100% sequence identity between the dsRNA and a gene transcript of a nucleic acid sequence of sequences with odd numbers of SEQ ID Nos in FIG. 1a, 1b, 1c or 1d or it's homolog is not necessarily required in order to bring about effective reduction in the expression. The advantage is, accordingly, that the method is tolerant with regard to sequence deviations as may be present as a consequence of genetic mutations, polymorphisms or evolutionary divergences. Thus, for example, using the dsRNA, which has been generated starting from a sequence of one of sequences of the nucleic acid as shown in FIG. 1a, 1b, 1c or 1d or homologs thereof of the one organism, may be used to suppress the corresponding expression in another organism.

Due to the high degree of sequence homology between the of FIG. 1a, 1b, 1c or 1d from various organisms (e.g. plants), i.e. proteins may be conserved to a high degree within, for example other plants, it is optionally possible that the expression of a dsRNA derived from one of the disclosed sequences as shown in FIG. 1a, 1b, 1c or 1d or homologs thereof also has an advantageous effect in other plant species.

The dsRNA can be synthesized either in vivo or in vitro. To this end, a DNA sequence encoding a dsRNA can be introduced into an expression cassette under the control of at least one genetic control element (such as, for example, promoter, enhancer, silencer, splice donor or splice acceptor or polyadenylation signal). Suitable advantageous constructs are described herein below. Polyadenylation is not required, nor do elements for initiating translation have to be present.

A dsRNA can be synthesized chemically or enzymatically. Cellular RNA polymerases or bacteriophage RNA polymerases (such as, for example T3, T7 or SP6 RNA polymerase) can be used for this purpose. Suitable methods for the in-vitro expression of RNA are described (WO 97/32016; U.S. Pat. No. 5,593,874; U.S. Pat. No. 5,698,425, U.S. Pat. No. 5,712,135, U.S. Pat. No. 5,789,214, U.S. Pat. No. 5,804,693). Prior to introduction into a cell, tissue or organism, a dsRNA which has been synthesized in vitro either chemically or enzymatically can be isolated to a higher or lesser degree from the reaction mixture, for example by extraction, precipitation, electrophoresis, chromatography or combinations of these methods. The dsRNA can be introduced directly into the cell or else be applied extracellularly (for example into the interstitial space).

Stable transformation of the plant with an expression construct which brings about the expression of the dsRNA is preferred, however. Suitable methods are described herein below.

B) Introduction of an Antisense Nucleic Acid Sequence

Methods for suppressing a specific protein by preventing the accumulation of its mRNA by means of “antisense” technology can be used widely and has been described extensively, including for plants (Sheehy et al. (1988) Proc. Natl. Acad. Sci. USA 85: 8805-8809; U.S. Pat. No. 4,801,100; Mol J N et al. (1990) FEBS Lett 268(2): 427-430). The antisense nucleic acid molecule hybridizes with, or binds to, the cellular mRNA and/or the genomic DNA encoding the target protein to be suppressed. This process suppresses the transcription and/or translation of the target protein. Hybridization can be brought about in the conventional manner via the formation of a stable duplex or, in the case of genomic DNA, by the antisense nucleic acid molecule-binding to the duplex of the genomic DNA by specific interaction in the large groove of the DNA helix.

An antisense nucleic acid sequence which is suitable for reducing the activity of a protein can be deduced using the nucleic acid sequence encoding this protein, for example the nucleic acid sequence as shown in FIG. 1a, 1b, 1c or 1d (or homologs, analogs, paralogs, orthologs thereof), by applying the base-pair rules of Watson and Crick. The antisense nucleic acid sequence can be complementary to all of the transcribed mRNA of the protein; it may be limited to the coding region, or it may only consist of one oligonucleotide which is complementary to part of the coding or noncoding sequence of the mRNA. Thus, for example, the oligonucleotide can be complementary to the nucleic acid region which encompasses the translation start for the protein. Antisense nucleic acid sequences may have an advantageous length of, for example, 5, 10, 15, 20, 25, 30, 35, 40, 45 or 50 nucleotides but they may also be longer and encompass at least 100, 200, 500, 1000, 2000 or 5000 nucleotides. Preferred is a length of 15-35, more preferred a length of 15, 20, 2, 30 or 35 nucleotides. Antisense nucleic acid sequences can be expressed recombinantly or synthesized chemically or enzymatically using methods known to the skilled worker. In the case of chemical synthesis, natural or modified nucleotides may be used. Modified nucleotides may confer increased biochemical stability to the antisense nucleic acid sequence and lead to an increased physical stability of the duplex formed by antisense nucleic acid sequence and sense target sequence. Examples of substances which can be used are phosphorothioate derivatives and acridine-substituted nucleotides such as 5-fluorouracil, 5-bromouracil, 5-chlorouracil, 5-iodouracil, hypoxanthin, xanthin, 4-acetyl-cytosine, 5-(carboxyhydroxymethyl)uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-man-nosylqueosine, 5′-methoxycarboxymethyluracil, 5-methoxyuracil, 2-methylthio-N6-iso-pentenyladenine, uracil-5-oxyacetic acid, pseudouracil, queosine, 2-thiocytosine, 5-methyl-2-thiouracil, 2-thiouracil, 4-thiouracil, 5-methyluracil, methyl uracil-5-oxyacetate, uracil-5-oxyacetic acid, 5-methyl-2-thiouracil, 3-(3-amino-3-N-2-carboxypropyl)uracil and 2,6-diaminopurine.

In a further preferred embodiment, the expression of a protein encoded by one of sequences of FIG. 1a, 1b, 1c or 1d or homologs, analogs, paralogs, orthologs thereof can be inhibited by nucleotide sequences which are complementary to the regulatory region of a gene (for example a promoter and/or enhancer) and which form triplex structures with the DNA-double helix in this region so that the transcription of the gene is reduced. Such methods have been described (Helene C (1991) Anticancer Drug Res. 6(6): 569-84; Helene C et al. (1992) Ann. NY Acad. Sci. 660: 27-36; Maher U (1992) Bioassays 14(12): 807-815).

In a further embodiment, the antisense nucleic acid molecule can be an α-anomeric nucleic acid. Such x-anomeric nucleic acid molecules form specific double-stranded hybrids with complementary RNA in which—as opposed to the conventional β-nucleic acids—the two strands run in parallel with one another (Gautier C et al. (1987) Nucleic Acids Res. 15: 6625-6641). Furthermore, the antisense nucleic acid molecule can also comprise 2′-O-methylribonucleotides (Inoue et al. (1987) Nucleic Acids Res. 15: 6131-6148) or chimeric RNA-DNA analogs (Inoue et al. (1987) FEBS Lett 215: 327-330).

The antisense nucleic acid molecules of the invention are typically administered to a cell or generated in situ such that they hybridize with or bind to cellular mRNA and/or genomic DNA encoding a polypeptide having the biological activity of protein of the invention thereby inhibit expression of the protein, e.g., by inhibiting transcription and/or translation and leading to the aforementioned compound X increasing activity.

The antisense molecule of the present invention comprises also a nucleic acid molecule comprising a nucleotide sequences complementary to the regulatory region of an nucleotide sequence encoding the natural occurring polypeptide of the invention, e.g. the polypeptide sequences shown in the sequence listing, or identified according to the methods described herein, e.g., its promoter and/or enhancers, e.g. to form triple helical structures that prevent transcription of the gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

C) Introduction of an Antisense Nucleic Acid Sequence Combined with a Ribozyme

It is advantageous to combine the above-described antisense strategy with a ribozyme method. Catalytic RNA molecules or ribozymes can be adapted to any target RNA and cleave the phosphodiester backbone at specific positions, thus functionally deactivating the target RNA (Tanner N K (1999) FEMS Microbiol. Rev. 23(3): 257-275). The ribozyme per se is not modified thereby, but is capable of cleaving further target RNA molecules in an analogous manner, thus acquiring the properties of an enzyme. The incorporation of ribozyme sequences into “antisense” RNAs imparts this enzyme-like RNA-cleaving property to precisely these “antisense” RNAs and thus increases their efficiency when inactivating the target RNA. The preparation and the use of suitable ribozyme “antisense” RNA molecules is described, for example, by Haseloff et. al. (1988) Nature 310: 585-591.

In this manner, ribozymes [for example “Hammerhead” ribozymes; Haselhoff and Gerlach (1988) Nature 310: 585-591] can be used to catalytically cleave the mRNA of an enzyme to be suppressed and to prevent translation. The ribozyme technology can increase the efficacy of an antisense strategy. Methods for expressing ribozymes for reducing specific proteins are described in (EP 0 291 533, EP 0 321 201, EP 0 360 257). Ribozyme expression has also been described for plant cells (Steinecke P et al. (1992). EMBO J 11(4): 1525-1530; de Feyter R et al. (1996) Mol. Gen. Genet. 250(3): 329-338). Suitable target sequences and ribozymes can be identified for example as described by Steinecke P, Ribozymes, Methods in Cell Biology 50, Galbraith et al. eds, Academic Press, Inc. (1995), pp. 449-460 by calculating the secondary structures of ribozyme RNA and target RNA and by their interaction [Bayley C C et al. (1992) Plant Mol. Biol. 18(2): 353-361; Lloyd A M and Davis R W et al. (1994) Mol. Gen. Genet. 242(6): 653-657]. For example, derivatives of the tetrahymena L-19 IVS RNA which have complementary regions to the mRNA of the protein to be suppressed can be constructed (see also U.S. Pat. No. 4,987,071 and U.S. Pat. No. 5,116,742). As an alternative, such ribozymes can also be identified from a library of a variety of ribozymes via a selection process (Bartel D and Szostak J W (1993) Science 261: 1411-1418).

D) Introduction of a (Sense) Nucleic Acid Sequence for Inducing Cosuppression

The expression of a nucleic acid sequence in sense orientation can lead to cosuppression of the corresponding homologous, endogenous genes. The expression of sense RNA with homology to an endogenous gene can reduce or indeed eliminate the expression of the endogenous gene, in a similar manner as has been described for the following antisense approaches: Jorgensen et al. [(1996) Plant Mol. Biol. 31 (5): 957-973], Goring et al. [(1991) Proc. Natl. Acad. Sci. USA 88: 1770-1774], Smith et al. [(1990) Mol. Gen. Genet. 224: 447-481], Napoli et al. [(1990) Plant Cell 2: 279-289] or Van der Krol et al. [(1990) Plant Cell 2: 291-99]. In this context, the construct introduced may represent the homologous gene to be reduced either in full or only in part. The application of this technique to plants has been described for example by Napoli et al. [(1990) The Plant Cell 2: 279-289 and in U.S. Pat. No. 5,010,323].

E) Introduction of Nucleic Acid Sequences Encoding a Dominant-Negative Protein

The function or activity of a protein can efficiently also be reduced by expressing a dominant-negative variant of said protein. The skilled worker is familiar with methods for reducing the function or activity of a protein by means of coexpression of its dominant-negative form [Lagna G and Hemmati-Brivanlou A (1998) Current Topics in Developmental Biology 36: 75-98; Perlmutter R M and Alberola-Ila J (1996) Current Opinion in Immunology 8(2): 285-90; Sheppard D (1994) American Journal of Respiratory Cell & Molecular Biology 11(1): 1-6; Herskowitz I (1987) Nature 329 (6136): 219-22].

A dominant-negative variant can be realized for example by changing of an amino acid in the proteins encoded by one of sequences of FIG. 1a, 1b, 1c or 1d or homologs thereof. This change can be determined for example by computer aided comparison (“alignment”). These mutations for achieving a dominant-negative variant are preferably carried out at the level of the nucleic acid sequences. A corresponding mutation can be performed for example by PCR-mediated in-vitro mutagenesis using suitable oligonucleotide primers by means of which the desired mutation is introduced. To this end, methods are used with which the skilled worker is familiar. For example, the “LA PCR in vitro Mutagenesis Kit” (Takara Shuzo, Kyoto) can be used for this purpose. It is also possible and known to those skilled in the art that deleting or changing of functional domains, e.g. TF or other signaling components which can bind but not activate may achieve the reduction of protein activity.

F) Introduction of DNA- or Protein-Binding Factors Against Genes, RNAs or Proteins

A reduction in the expression of a gene encoded by one of sequences of FIG. 1a, 1b, 1c or 1d or homologs thereof according to the invention can also be achieved with specific DNA-binding factors, for example factors of the zinc finger transcription factor type. These factors attach to the genomic sequence of the endogenous target gene, preferably in the regulatory regions, and bring about repression of the endogenous gene. The use of such a method makes possible the reduction in the expression of an endogenous gene without it being necessary to recombinantly manipulate the sequence of the latter. Such methods for the preparation of relevant factors are described in Dreier B et al. [(2001) J. Biol. Chem. 276(31): 29466-78 and (2000) J. Mol. Biol. 303(4): 489-502], Beerli R R et al. [(1998) Proc. Natl. Acad. Sci. USA 95(25): 14628-14633; (2000) Proc. Natl. Acad. Sci. USA 97(4): 1495-1500 and (2000) J. Biol. Chem. 275(42): 32617-32627)], Segal D J and Barbas C F [3rd (2000) Curr. Opin. Chem. Biol. 4(1): 10-39], Kang J S and Kim J S [(2000) J. Biol. Chem. 275(12): 8742-8748], Kim J S et al. [(1997) Proc. Natl. Acad. Sci. USA 94(8): 3616-3620], Klug A [(1999) J. Mol. Biol. 293(2): 215-218], Tsai S Y et al. [(1998) Adv. Drug Deliv. Rev. 30(1-3): 23-31], Mapp A K et al. [(2000) Proc. Natl. Acad. Sci. USA 97(8): 3930-3935], Sharrocks A D et al. [(1997) Int. J. Biochem. Cell Biol. 29(12): 1371-1387] and Zhang L et al. [(2000) J. Biol. Chem. 275(43): 33850-33860]. Examples for the application of this technology in plants have been described in WO 01/52620, Ordiz M I et al., (Proc. Natl. Acad. Sci. USA, Vol. 99, Issue 20, 13290-13295, 2002) or Guan et al., (Proc. Natl. Acad. Sci. USA, Vol. 99, Issue 20, 13296-13301, 2002)

These factors can be selected using any portion of a gene. This segment is preferably located in the promoter region. For the purposes of gene suppression, however, it may also be located in the region of the coding exons or introns. The skilled worker can obtain the relevant segments from Genbank by database search or starting from a cDNA whose gene is not present in Genbank by screening a genomic library for corresponding genomic clones.

It is also possible to first identify sequences in a target crop which are encoded by one of sequences of FIG. 1a, 1b, 1c or 1d or homologs thereof, then find the promoter and reduce expression by the use of the above mentioned factors.

The skilled worker is familiar with the methods required for doing so.

Furthermore, factors which are introduced into a cell may also be those which themselves inhibit the target protein. The protein-binding factors can, for example, be aptamers [Famulok M and Mayer G (1999) Curr. Top Microbiol. Immunol. 243: 123-36] or antibodies or antibody fragments or single-chain antibodies. Obtaining these factors has been described, and the skilled worker is familiar therewith. For example, a cytoplasmic scFv antibody has been employed for modulating activity of the phytochrome A protein in genetically modified tobacco plants [Owen M et al. (1992) Biotechnology (NY) 10(7): 790-794; Franken E et al. (1997) Curr. Opin. Biotechnol. 8(4): 411-416; Whitelam (1996) Trend Plant Sci. 1: 286-272].

Gene expression may also be suppressed by tailor-made low-molecular-weight synthetic compounds, for example of the polyamide type [Dervan P B and Bürli R W (1999) Current Opinion in Chemical Biology 3: 688-693; Gottesfeld J M et al. (2000) Gene Expr. 9(1-2): 77-91]. These oligomers consist of the units 3-(dimethylamino)propylamine, N-methyl-3-hydroxypyrrole, N-methylimidazole and N-methylpyrroles; they can be adapted to each portion of double-stranded DNA in such a way that they bind sequence-specifically to the large groove and block the expression of the gene sequences located in this position. Suitable methods have been described in Bremer R E et al. [(2001) Bioorg. Med. Chem. 9(8): 2093-103], Ansari A Z et al. [(2001) Chem. Biol. 8(6): 583-92], Gottesfeld J M et al. [(2001) J. Mol. Biol. 309(3): 615-29], Wurtz N R et al. [(2001) Org. Lett 3(8): 1201-3], Wang C C et al. [(2001) Bioorg. Med. Chem. 9(3): 653-7], Urbach A R and Dervan P B [(2001) Proc. Natl. Acad. Sci. USA 98(8): 4103-8] and Chiang S Y et al. [(2000) J. Biol. Chem. 275(32): 24246-54].

G) Introduction of Viral Nucleic Acid Sequences and Expression Constructs which Bring about the Degradation of RNA

Inactivation or downregulation can also be efficiently brought about by inducing specific RNA degradation by the organism, advantageously in the plant, with the aid of a viral expression system (Amplikon) [Angell, S M et al. (1999) Plant J. 20(3): 357-362]. Nucleic acid sequences with homology to the transcripts to be suppressed are introduced into the plant by these systems—also referred to as “VIGS” (viral induced gene silencing) with the aid of viral vectors. Then, transcription is switched off, presumably mediated by plant defense mechanisms against viruses. Suitable techniques and methods are described in Ratcliff F et al. [(2001) Plant J. 25(2): 237-45], Fagard M and Vaucheret H [(2000) Plant Mol. Biol. 43(2-3): 285-93], Anandalakshmi R et al. [(1998) Proc. Natl. Acad. Sci. USA 95(22): 13079-84] and Ruiz M T [(1998) Plant Cell 10(6): 937-46].

H) Introduction of Constructs for Inducing a Homologous Recombination on Endogenous Genes, for Example for Generating Knock-Out Mutants

To generate a homologously-recombinant organism with reduced activity, a nucleic acid construct is used which, for example, comprises at least part of an endogenous gene which is modified by a deletion, addition or substitution of at least one nucleotide in such a way that the functionality is reduced or completely eliminated. The modification may also affect the regulatory elements (for example the promoter) of the gene so that the coding sequence remains unmodified, but expression (transcription and/or translation) does not take place and is reduced.

In the case of conventional homologous recombination, the modified region is flanked at its 5′ and 3′ end by further nucleic acid sequences which must be sufficiently long for allowing recombination. Their length is, as a rule, in a range of from one hundred bases up to several kilobases [Thomas K R and Capecchi M R (1987) Cell 51: 503; Strepp et al. (1-998) Proc. Natl. Acad. Sci. USA 95(8): 4368-4373]. In the case of homologous recombination, the host organism—for example a plant—is transformed with the recombination construct using the methods described herein below, and clones which have successfully undergone recombination are selected using for example a resistance to antibiotics or herbicides. Using the cotransformation technique, the resistance to antibiotics or herbicides can subsequently advantageously be re-eliminated by performing crosses. An example for an efficient homologous recombination system in plants has been published in Nat. Biotechnol. 2002 October; 20(10):1030-4, Terada R et al.: Efficient gene targeting by homologous recombination in rice.

Homologous recombination is a relatively rare event in higher eukaryotes, especially in plants. Random integrations into the host genome predominate. One possibility of removing the randomly integrated sequences and thus increasing the number of cell clones with a correct homologous recombination is the use of a sequence-specific recombination system as described in U.S. Pat. No. 6,110,736, by means of which unspecifically integrated sequences can be deleted again, which simplifies the selection of events which have integrated successfully via homologous recombination. A multiplicity of sequence-specific recombination systems may be used, examples which may be mentioned being Cre/lox system of bacteriophage P1, the FLP/FRT system from yeast, the Gin recombinase of phage Mu, the Pin recombinase from E. coli and the R/RS system of the pSR1 plasmid. The bacteriophage P1 Cre/lox system and the yeast FLP/FRT system are preferred. The FLP/FRT and the cre/lox recombinase system have already been applied to plant systems [Odell et al. (1990) Mol. Gen. Genet. 223: 369-378].

I) Introduction of Mutations into Endogenous Genes for Bringing about a Loss of Function (for Example Generation of Stop Codons, Reading-Frame Shifts and the Like)

Further suitable methods for reducing activity are the introduction of nonsense mutations into endogenous genes, for example by introducing RNA/DNA oligonucleotides into the plant [Zhu et al. (2600) Nat. Biotechnol. 18(5): 555-558], and the generation of knock-out mutants with the aid of, for example, T-DNA mutagenesis [Koncz et al. (1992) Plant Mol. Biol. 20(5): 963-976], ENU-(N-ethyl-N-nitrosourea)-mutagenesis or homologous recombination [ENU-(N-ethyl-N-nitrosourea)-mutagenesis or homologous recombination [Hohn B and Puchta (1999) H. Proc. Natl. Acad. Sci. USA 96: 8321-8323]. Point mutations may also be generated by means of DNA-RNA hybrids also known as “chimeraplasty” [Cole-Strauss et al. (1999) Nucl. Acids Res. 27(5): 1323-1330; Kmiec (1999) Gene Therapy American Scientist 87(3): 240-247]. The mutation sites may be specifically targeted or randomly selected.

Nucleic acid sequences as described in item B) to I) are expressed in the cell or organism by transformation/transfection of the cell or organism or are introduced in the cell or organism by known methods, for example as disclosed in item A).

Other suitable method for reducing activity is the introduction of a nucleic acid in the plant cell, which interacts with a gene encoded by one or more nucleic acid sequences selected from the group consisting of sequences of FIG. 1a, 1b, 1c or 1d and/or homologs thereof. The interaction of the introduced nucleic acid, which can be active itself, with said gene leads by deletion, inversion or insertion finally to inactivation, i.e. by frameshift, or destruction of said gene.

In particular, the invention provides a method of producing a transformed plant with a gene encoding nucleic acid, wherein inactivation or down-regulation of said gene(s) in the plant results in increased tolerance to environmental stress, which is preferably achieved by altering metabolic activity, as compared to a wild type plant, comprising the inactivation or down-regulation by mutation of a nucleic acid sequence of FIG. 1a, 1b, 1c or 1d or homologs thereof.

For such plant transformation, binary vectors such as pBinAR can be used (Höfgen and Willmitzer, 1990Plant Science 66:221-230). Moreover suitable binary vectors are such as pBIN19, pBI101, pGPTV or pPZP (Hajukiewicz, P. et al., 1994, Plant Mol. Biol., 25: 989-994). An overview of binary vectors and their specific features is given in Hellens et al., 2000, Trends in plant science, 5: 446-451.

Construction of the binary vectors can be performed by ligation of the cDNA in sense or antisense orientation into the T-DNA. 5-prime to the cDNA a plant promoter activates transcription of the cDNA. A polyadenylation sequence is located 3-prime to the cDNA. Tissue-specific expression can be achieved by using a tissue specific promoter as listed below. Also, any other promoter element can be used. For constitutive expression within the whole plant, the CaMV 35S promoter can be used. The expressed protein can be targeted to a cellular compartment using a signal peptide, for example for plastids, mitochondria or endoplasmic reticulum (Kermode, 1996 Crit. Rev. Plant Sci. 4(15):285-423). The signal peptide is cloned 5-prime in frame to the cDNA to archive subcellular localization of the fusion protein. Additionally, promoters that are responsive to abiotic stresses can be used with, such as the Arabidopsis promoter RD29A. One skilled in the art will recognize that the promoter used should be operatively linked to the nucleic acid such that the promoter causes transcription of the nucleic acid which results in the synthesis of a mRNA which encodes a polypeptide. Alternatively, the RNA can be an antisense RNA for use in affecting subsequent expression of the same or another gene or genes.

Alternate methods of transfection include the direct transfer of DNA into developing flowers via electroporation or Agrobacterium mediated gene-transfer. Agrobacterium mediated plant transformation can be performed using for example the GV3101 (pMP90) (Koncz and Schell, 1986 Mol. Gen. Genet. 204:383-396) or LBA4404 (Ooms et al., Plasmid, 1982, 7: 15-29; Hoekema et al., Nature, 1983, 303: 179-180) Agrobacterium tumefaciens strain. Transformation can be performed by standard transformation and regeneration techniques (Deblaere et al., 1994 Nucl. Acids. Res. 13:4777-4788; Gelvin and Schilperoort, Plant Molecular Biology Manual, 2nd Ed.—Dordrecht: Kluwer Academic Publ., 1995.—in Sect., Ringbuch Zentrale Signatur: BT11-P ISBN 0-7923-2731-4; Glick, B R and Thompson, J E, Methods in Plant Molecular Biology and Biotechnology, Boca Raton: CRC Press, 1993.—360 S., ISBN 0-8493-5164-2). For example, rapeseed can be transformed via cotyledon or hypocotyl transformation (Moloney et al., 1989 Plant Cell Reports 8:238-242; De Block et al., 1989 Plant Physiol. 91:694-701). Use of antibiotics for Agrobacterium and plant selection depends on the binary vector and the Agrobacterium strain used for transformation. Rapeseed selection is normally performed using kanamycin as selectable plant-marker. Agrobacterium mediated gene transfer to flax can be performed using, for example, a technique described by Mlynarova et al., 1994 Plant Cell Report 13:282-285. Additionally, transformation of soybean can be performed using for example a technique described in European Patent No. 0424 047, U.S. Pat. No. 5,322,783, European Patent No. 0397 687, U.S. Pat. No. 5,376,543 or U.S. Pat. No. 5,169,770. Transformation of maize can be achieved by particle bombardment, polyethylene glycol mediated DNA uptake or via the silicon carbide fiber technique. (See, for example, Freeling and Walbot “The maize handbook” Springer Verlag: New York (1993) ISBN 3-540-97826-7). A specific example of maize transformation is found in U.S. Pat. No. 5,990,387 and a specific example of wheat transformation can be found in PCT Application No. WO 93/07256.

In particular, a useful method to ascertain the level of transcription or activity of the gene (an indicator of the amount of mRNA available for translation to the gene product) is to perform a Northern blot (for reference see, for example, Ausubel et al., 1988 Current Protocols in Molecular Biology, Wiley: New York). This information at least partially demonstrates the degree of transcription of the transformed gene. Total cellular RNA can be prepared from cells, tissues or organs by several methods, all well-known in the art, such as that described in Bormann, E. R. et al., 1992 Mol. Microbiol. 6:317-326. To assess the presence or relative quantity of protein translated from this mRNA, standard techniques, such as a Western blot, may be employed. These techniques are well known to one of ordinary skill in the art. (See for example Ausubel et al., 1988 Current Protocols in Molecular Biology, Wiley: New York). The use of Real Time PCR is also possible.

The invention may further be combined with an isolated recombinant expression vector comprising a stress related protein encoding nucleic acid, wherein expression of the vector or stress related protein encoding nucleic acid, respectively in a host cell results in increased tolerance and/or resistance to environmental stress, which is preferably achieved by altering metabolic activity, as compared to the wild type of the host cell. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses), which serve equivalent functions.

A plant expression cassette comprising a nucleic acid construct, which when expressed allows inactivation or down-regulation of a gene encoded by a nucleic acid selected from the group consisting of sequences of FIG. 1a, 1b, 1 c or 1d and/or homologs thereof and/or parts thereof by a method mentioned above leading to increased stress tolerance and/or resistance, which is preferably achieved by altering metabolic activity, is also included in the scope of the present invention.

The plant expression cassette preferably contains regulatory sequences capable of driving gene expression in plant cells and operably linked so that each sequence can fulfill its function, for example, termination of transcription by polyadenylation signals. Preferred polyadenylation signals are those originating from Agrobacterium tumefaciens T-DNA such as the gene 3 known as octopine synthase of the Ti-plasmid pTiACH5 (Gielen et al., 1984 EMBO J. 3:835) or functional equivalents thereof but also all other terminators functionally active in plants are suitable.

Plant gene expression must be operably linked to an appropriate promoter conferring gene expression in a time, cell or tissue specific manner. Preferred promoters are such that drive constitutive expression (Benfey et al., 1989 EMBO J. 8:2195-2202) like those derived from plant viruses like the 35S CaMV (Franck et al., 1980 Cell 21:285-294), the 19S CaMV (see also U.S. Pat. No. 5,352,605 and PCT Application No. WO 8402913) or plant promoters like those from Rubisco small subunit described in U.S. Pat. No. 4,962,028.

Additional advantageous regulatory sequences are, for example, included in the plant promoters such as CaMV/35S [Franck et al., Cell 21 (1980) 285-294], PRP1 [Ward et al., Plant. Mol. Biol. 22 (1993)], SSU, OCS, lib4, usp, STLS1, B33, LEB4, nos or in the ubiquitin, napin or phaseolin promoter. Additional useful plant promoters are the cytosolic FBPase promoter or ST-LSI promoter of the potato (Stockhaus et al., EMBO J. 8, 1989, 2445), the phosphorybosyl phyrophoshate amido transferase promoter of Glycine max (gene bank accession No. U87999) or the noden specific promoter described in EP-A-0 249 676. Additional particularly advantageous promoters are seed specific promoters which can be used for monocotyledons or dicotyledons. Described in U.S. Pat. No. 5,608,152 (napin promoter from rapeseed), WO 98/45461 (phaseolin promoter from Arobidopsis), U.S. Pat. No. 5,504,200 (phaseolin promoter from Phaseolus vulgaris), WO 91/13980 (Bce4 promoter from Brassica) and Baeumlein et al., Plant J., 2, 2, 1992: 233-239 (LEB4 promoter from leguminosa) are promoters useful in dicotyledons. The following promoters are useful for example in monocotyledons Ipt-2- or Ipt-1-promoter from barley (WO 95/15389 and WO 95/23230) or hordein promoter from barley. Other useful promoters described in WO 99/16890.

It is possible in principle to inactivate or down-regulate all natural promoters with their regulatory sequences like those mentioned above in order to e.g. reduce the level of production of a targeted protein.

The construct may also comprise further genes which are to be inserted into the organisms and which are for example involved in stress resistance, i.e. next to inactivating certain genes or incorporating inactivated genes at their place, it is possible to introduce favorable genes that are related to production of proteins which actively increase stress tolerance or resistance. It is therefore feasible and advantageous to insert and express in host organisms regulatory genes such as genes for inducers, repressors or enzymes which intervene by their enzymatic activity in the regulation of one or more or all genes of a biosynthetic pathway. These genes can be heterologous or homologous in origin. The inserted genes may have their own promoter or else be under the control of same promoter as sequences of FIG. 1a, 1b, 1c or 1d or their homologs.

The gene construct advantageously comprises, for expression of the other genes present, additionally 3′ and/or 5′ terminal regulatory sequences to enhance expression, which are selected for optimal expression depending on the selected host organism and gene or genes.

These regulatory sequences are intended to make specific expression of the genes and protein expression possible as mentioned above. This may mean, depending on the host organism, for example that the gene is expressed or overexpressed only after induction, or that it is immediately expressed and/or overexpressed.

The regulatory sequences or factors may moreover preferably have a beneficial effect on expression of the introduced genes, and thus increase it. It is possible in this way for the regulatory elements to be enhanced advantageously at the transcription level by using strong transcription signals such as promoters and/or enhancers. However, in addition, it is also possible to enhance translation by, for example, improving the stability of the mRNA.

Other preferred sequences for use in plant gene expression cassettes are targeting-sequences necessary to direct the gene product in its appropriate cell compartment (for review see Kermode, 1996 Crit. Rev. Plant Sci. 15(4):285-423 and references cited therein) such as the vacuole, the nucleus, all types of plastids like amyloplasts, chloroplasts, chromoplasts, the extracellular space, mitochondria, the endoplasmic reticulum, oil bodies, peroxisomes and other compartments of plant cells.

TABLE 1
Examples of Tissue-specific and Stress
inducible promoters in plants

| Expression | Reference |
|---|---|
| Cor78- Cold, drought, | Ishitani, et al., Plant Cell 9: 1935-1949 |
| salt, ABA, wounding- | (1997). Yamaguchi-Shinozaki and |
| inducible | Shinozaki, Plant Cell 6: 251-264 (1994). |
| Rci2A - Cold, | Capel et al., Plant Physiol 115: 569-576 |
| dehydration-inducible | (1997) |
| Rd22 - Drought, salt | Yamaguchi-Shinozaki and Shinozaki, Mol |
| | Gen Genet 238: 17-25 (1993). |
| Cor15A - Cold, | Baker et al., Plant Mol. Biol. 24: 701-713 |
| dehydration, ABA | (1994). |
| GH3- Auxin inducible | Liu et al., Plant Cell 6: 645-657 (1994) |
| ARSK1-Root, salt | Hwang and Goodman, Plant J 8: 37-43 |
| inducible | (1995). |
| PtxA - Root, salt | GenBank accession X67427 |
| inducible | |
| SbHRGP3 - Root specific | Ahn et al., Plant Cell 8: 1477-1490 (1998). |
| KST1 - Guard cell | Plesch et al., Plant Journal 28(4): 455-64 |
| specific | (2001). |
| KAT1 - Guard cell | Plesch et al., Gene 249: 83-89 (2000) |
| specific | Nakamura et al., Plant Physiol. 109: |
| | 371-374 (1995) |
| salicylic acid inducible | PCT Application No. WO 95/19443 |
| tetracycline inducible | Gatz et al. Plant J. 2: 397-404 (1992) |
| Ethanol inducible | PCT Application No. WO 93/21310 |
| pathogen inducible PRP1 | Ward et al., 1993 Plant. Mol. Biol. 22: |
| | 361-366 |
| heat inducible hsp80 | U.S. Pat. No. 5187267 |
| cold inducible alpha- | PCT Application No. WO 96/12814 |
| amylase | |
| Wound-inducible pinll | European Patent No. 375091 |
| RD29A - salt-inducible | Yamaguchi-Shinozalei et al. (1993) Mol. |
| | Gen. Genet. 236: 331-100 |
| plastid-specific viral | PCT Application No. WO 95/16783 and |
| RNA-polymerase | WO 97/06250 |

Selection marker systems, like the AHAS marker or other promoters, e.g. superpromotor (Ni et al., Plant Journal 7, 1995: 661-676), Ubiquitin promoter (Callis et al., J. Biol. Chem., 1990, 265: 12486-12493; U.S. Pat. No. 5,510,474; U.S. Pat. No. 6,020,190; Kawalleck et al., Plant. Molecular Biology, 1993, 21: 673-684) or 10S promoter (GenBank Accession numbers M59930 and X16673) may be similar useful for the combination with the present invention and are known to a person skilled in the art.

In particular, the present invention describes using the altered metabolic activity by inactivation or down-regulation of genes to engineer stress-tolerant and/or resistant, i.e. drought-, salt- and/or cold-tolerant and/or resistant plants. This strategy has herein been demonstrated for Arabidopsis thaliana, but its application is not restricted to these plants. Accordingly, the invention provides a transformed plant containing one or more (stress related protein encoding) genes selected from sequences of FIG. 1a, 1b, 1c or 1d or homologs thereof, that are inactivated or down-regulated and stress tolerance and/or resistance, which is preferably achieved by altering metabolic activity, wherein the (environmental) stress is drought, increased salt or decreased or increased temperature but its application is not restricted to these adverse environments. Protection against other adverse conditions such as heat, air pollution, heavy metals and chemical toxicants, for example, may be obtained. In particularly preferred embodiments, the environmental stress is drought.

Growing the modified plants under stress conditions and then screening and analyzing the growth characteristics and/or metabolic activity assess the effect of the genetic modification in plants on stress tolerance and/or resistance. Such analysis techniques are well known to one skilled in the art. They include next to screening (Römpp Lexikon Biotechnologie, Stuttgart/New York: Georg Thieme Verlag 1992, “screening” p. 701) dry weight, wet weight, protein synthesis, carbohydrate synthesis, lipid synthesis, evapotranspiration rates, general plant and/or crop yield, flowering, reproduction, seed setting, root growth, respiration rates, photosynthesis rates, etc. (Applications of HPLC in Biochemistry in: Laboratory Techniques in Biochemistry and Molecular Biology, vol. 17; Rehm et al., 1993 Biotechnology, vol. 3, Chapter III: Product recovery and purification, page 469-714, VCH: Weinheim; Belter, P. A. et al., 1988 Bioseparations: downstream processing for biotechnology, John Wiley and Sons; Kennedy, J. F. and Cabral, J. M. S., 1992 Recovery processes for biological materials, John Wiley and Sons; Shaeiwitz, J. A. and Henry, J. D., 1988 Biochemical separations, in: Ulmann's Encyclopedia of Industrial Chemistry, vol. B3, Chapter-11, page 1-27, VCH: Weinheim; and Dechow, F. J. (1989) Separation and purification techniques in biotechnology, Noyes Publications).

The methods of the invention may be used to detect environmental stress in plant cells or plants by screening the plant cells for altered metabolic activity as compared to non-stress conditions, which allows for selection of resistant or tolerant plants or plant cells and also provides detection of stress in plants or plant cells before symptoms are visible and damage is high.

The methods of the invention also allow breeding of plant cells or plants towards increased tolerance and/or resistance to environmental stress by screening the plant cells under stress conditions for altered metabolic activity as compared to non-stress conditions and selecting those with increased tolerance and/or resistance to environmental stress for further replication.

The engineering of one or more stress related genes of the invention may also result in stress related proteins having altered activities which indirectly impact the stress response and/or stress tolerance of plants. For example, the normal biochemical processes of metabolism result in the production of a variety of products (e.g., hydrogen peroxide and other reactive oxygen species) which may actively interfere with these same metabolic processes (for example, peroxynitrite is known to react with tyrosine side chains, thereby inactivating some enzymes having tyrosine in the active site (Groves, J. T., 1999 Curr. Opin. Chem. Biol. 3(2):226-235). By optimizing the inactivation or down-regulation of one or more stress related genes of the invention, it may be possible to improve the metabolic activity leading to higher stress tolerance and/or resistance of the cell.

Additionally, the sequences disclosed herein, or fragments thereof, can be targeted to generate knockout mutations in the genomes of various other plant cells (Girke, T., 1998 The Plant Journal 15:39-48). The resultant knockout cells can then be evaluated for their ability or capacity to tolerate various stress conditions, their response to various stress conditions, and the effect on the phenotype and/or genotype of, the mutation. For other methods of gene inactivation see U.S. Pat. No. 6,004,804 “Non-Chimeric Mutational Vectors” and Puttaraju et al., 1999 Spliceosome-mediated RNA trans-splicing as a tool for gene therapy Nature Biotechnology 17:246-252.

Throughout this application, various publications are referenced. The disclosures of all of these publications and those references cited within those publications in their entireties are hereby incorporated by reference into this application in order to more fully describe the state of the art to which this invention pertains.

This invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLE

Engineering Stress-Tolerant Arabidopsis Plants with Altered Metabolic Activity by Inactivation or Down-Regulation Stress Related Genes.

Transformation of Arabidopsis thaliana

Vector Preparation

A binary vector was constructed based on the modified pPZP binary vector backbone (comprising the kanamycin-gene for bacterial selection; Hajdukiewicz, P. et al., 1994, Plant Mol. Biol., 25: 989-994) and the selection marker bar-gene (De Block et al., 1987, EMBO J. 6, 2513-2518) driven by the mas240 1′ and mas271f promoters (Velten et al., 1984, EMBO J. 3, 2723-2730; Mengiste, Amedeo and Paszkowski, 1997, Plant J., 12, 945-948). The complete vector is shown in FIG. 2.

Examples of other usable binary vectors for insertional mutagenesis are pBIN19, pBI101, pBinAR or pGPTV. An overview over binary vectors and their specific features is given in Hellens et al., 2000, Trends in plant Science, 5:446-451 and in Guerineau F., Mullineaux P., 1993, Plant transformation and expression vectors in plant molecular biology, LABFAX Series, (Croy R. R. D., ed.) pp. 121-127 Bios Scientific Publishers, Oxford.

Transformation of Agrobacteria

The plasmid was transformed into Agrobacterium tumefaciens (GV3101 pMP90; Koncz and Schell, 1986 Mol. Gen. Genet. 204:383-396) using heat shock or electroporation protocols. Transformed colonies were grown on YEB medium and selected by respective antibiotics (Rif/Gent/Km) for 2 d at 28° C. These agrobacteria cultures were used for the plant transformation.

Arabidopsis thaliana of the ecotype C24 were grown and transformed according to standard conditions (Bechtold, N., Ellis, J., Pelletier, G. 1993. In planta Agrobacterium mediated gene transfer by infiltration of Arabidopsis thaliana plants, C. R. Acad. Sci. Paris 316:1194-1199; Bent, A. F., Clough, J. C., 1998; Floral dip: a simplified method for Agrobacterium-mediated transformation of Arabidopsis thaliana, PLANT J. 16:735-743).

Transformed plants (F1) were selected by the use of their respective resistance marker. In case of BASTA®-resistance, plantlets were sprayed four times at an interval of 2 to 3 days with 0.02% BASTA® and transformed plants were allowed to set seeds. 50-100 seedlings (F2) were subjected again to marker selection, in case of BASTA-resistance by spaying with 0.1% BASTA® on 4 consecutive days during the plantlet phase. Plants segregating for a single resistance locus (approximately 3:1 resistant seedling to sensitive seedlings) were chosen or further analysis. From these lines three of the resistant seedlings (F2) were again allowed to set seeds and were tested for homozygosis through in-vitro germination of their seeds (F3) on agar medium containing the selection agent (BASTA®, 15 mg/L ammonium glufosinate, Pestanal, Riedel de Haen, Seelze, Germany). Those F2 lines which showed nearly 100% resistant offspring (F3) were considered homozygote and taken for functional analysis.

Measurement of Stress Tolerance

Transformed A. thaliana plants were grown individually in pots containing a 4:1 (v/v) mixture of soil and quartz sand in a growth chamber (York Industriekälte GmbH, Mannheim, Germany). To induce germination, sown seeds were kept at 4° C., in the dark, for 3 days. Standard growth conditions were: photoperiod of 16 h light and 8 h dark, 20° C., 60% relative humidity, and a photon flux density of 150 μE. Plants were watered daily until they were approximately 3 weeks old at which time drought was imposed by withholding water. Simultaneously, the relative humidity was reduced in 10% increments every second day to 20%. After approximately 12 days of withholding water, most plants showed visual symptoms of injury, such as wilting and leaf browning, whereas tolerant or resistant plants were identified as being visually turgid and healthy green in color. Plants were scored for symptoms of drought injury in comparison to wild type and neighboring plants for 3-5 days in succession.

Three successive experiments were conducted. In the first experiment, one individual of each transformed line was tested.

In the second experiment, the lines that had been scored as tolerant or resistant in the first experiment, i.e. survived longer than the wild type control, were put through a confirmation screen according to the same experimental procedures. In this experiment, max. 5 plants of each tolerant or resistant line were grown and treated as before.

In the first two experiments, resistance or tolerance was measured compared to neighboring and wild type plants.

In the third experiment (table 2), at least 15, but usually 20-25 replicates of each confirmed tolerant line, i.e. those that had been scored as tolerant or resistant in the second experiment, were grown and treated as before. In the third-experiment the average and maximum number of days of drought survival after the wild-type control had visually died was determined. Additionally measurements of chlorophyll fluorescence (table 3) were made in stressed and non-stressed plants using a Mini-PAM (Heinz Walz GrmbH, Effeltrich, Germany).

In the third experiment, after 12 days of drought, the control (non-transformed Arabidopsis thaliana) and most transformed lines in the test showed extreme visual symptoms of stress including necrosis and cell death. Several transformed plants retained viability as shown by their turgid appearance and maintenance of green color.

Chlorophyll fluorescence measurements of photosynthetic yield (in non-dark adapted plants) confirmed that 14 days of drought stress completely inhibited photosynthesis in the control plants. In most cases the transformed lines maintained photosynthetic function longer (table 3).

Analysis of the Selected Stress Resistant Lines

Since the lines were preselected for single insertion loci and a homozygous situation of the resistance marker, the disruption (or mutation) of single genes through the integration of the T-DNA were expected to have lead to the stress-resistant phenotype. Lines which showed a consistent phenotype were chosen for molecular analysis.

Genomic DNA was purified from approximately 100 mg of leaf tissue from these lines using standard procedures (either spins columns from Qiagen, Hilden, Germany or the Nucleon Phytopure Kit from Amersham Biosciences, Freiburg, Germany). The amplification of the insertion side of the T-DNA was achieved using two different methods. Either by an adaptor PCR-method according to Spertini D, Béliveau C. and Bellemare G., 1999, Biotechniques, 27, 308-314 using T-DNA specific primers LB1 (5′-TGA CGC CAT TTC GCC TTT TCA-3′; SEQ ID 284) for the first and LB2 (5′-CAG AAA TGG-ATA AAT AGC CTT GCT TCC-3′; SEQ ID 285) or RB4-2 (5′-AGC TGG CGT MT AGC GM GAG-3′; SEQ ID 286) for the second round of PCR. Alternatively TAIL-PCR (Liu Y-G, Mitsukawa N, Oosumi T and Whittier R F, 1995, Plant J. 8, 457-463 was preformed. In this case in the first round PCR LB1 (5′-TGA CGC CAT TTC GCC TTT TCA-3′ SEQ ID 284) or RB1-2 (5′-CAA CTT MT CGC CTT GCA GCA CA-3′; SEQ ID 287), for the second round LB2 (5′-CAG MA TGG ATA MT AGC CTT GCT TCC-3′ SEQ ID 285) or RB4-2 (5′-AGC TGG CGT MT AGC GM GAG-3′ SEQ ID 286) and in the last round LB3 (5′-CCA ATA CAT TAC ACT AGC ATC TG-3′; SEQ ID 288) or RB5 (5′-MT GCT AGA GCA GCT TGA-3′; SEQ ID 289) were used as T-DNA specific primers for left or right T-DNA borders respectively.

Appropriate PCR-products were identified on agarose gels and purified using columns and standard procedures (Qiagen, Hilden, Germany). PCR-products were sequenced with additional T-DNA-specific primers located towards the borders relative to the primers used for amplification. For PCR products containing left border sequences primer LBseq (5′-CM TAC ATT ACA CTA GCA TCT G-3′; SEQ ID 290) and for sequences containing right border sequences primer RBseq (5′-AGA GGC CCG CAC CGA TCG-3′; SEQ ID 291) was used for sequencing reactions. The resulting sequences were taken for comparison with the available Arabidopsis genome sequence from Genbank using the blast algorithm (Altschul et al., 1990. J Mol Biol, 215:403-410).

Details on PCR products used to identify the genomic locus are given in table 4. Indicated are the identified annotated open reading frame in the Arabidopsis genome, the estimated size of the obtained PCR product (in base pairs), the T-DNA border (LB: left border, RB: right border) for which the amplification was achieved, the method which resulted in the indicated PCR product (explanation see text above), the respective restriction enzymes in case of adaptor PCR, and the degenerated primer in the case of TAIL PCR. Routinely degenerated primers ADP3 (5′-WGTGNAGWANCANAGA-3′; SEQ ID 292), ADP6 (5′-AGWGNAGWANCANAGA-3′; SEQ ID 293) and ADP8 (5′-NTGCGASWGANWAGAA-3′; SEQ ID 294) were used next to the other known primers given in table 4.

The identification of the insertion locus in each case was confirmed by a control PCR, using a T-DNA-specific primer and a primer deduced from the identified genomic locus, near to the insertion side. The amplification of a PCR-product of the expected size from the insertion line using these two primers proved the disruption of the identified locus by the T-DNA integration.

Metabolic Analysis of Transgenic Plants

The described metabolic changes in transgenic plants were identified using the following experimental procedure:

a) Growth and Treatment of Plants

Plants were grown in climate chambers under standard conditions on pot soil for three weeks (see above). Eight days prior to harvest, water was withheld for part of the plants (8-day treatment). Four days prior to harvest, water was withheld, for another group of plants (4-day treatment). The plants of “control treatment” were normally watered throughout the growth period. Plants due to be analysed in, the same analytical sequence were grown side-by-side to avoid environmental influences.

b) Sampling and Storage of Samples

Sampling took place in the climate chamber. Green parts were cut with a pair of scissors, quickly weighed, and immediately put into a liquid nitrogen pre-cooled extraction thimble. Racks with extraction thimbles were stored at −80° C. until extraction.

c) Freeze-Drying

Plants were not allowed to thaw or reach temperatures >−40° C. until either the first contact with solvents or the removal of water by freeze-drying.

The sample rack with extraction thimbles was put into the pre-cooled (−40° C.) freeze-dryer. The starting temperature for the main drying phase was −35° C., pressure was 0.120 mbar. For the drying process, parameters were changed according to a pressure and temperature program. The final temperature (after 12 hours) was +30° C., pressure was 0.001-0.004 mbar. After shutting down the vacuum pump and cooling machine, the system was aired with dried air or Argon.

d) Extraction

Extraction thimbles with plant material were transferred to 5 mL extraction cells on the ASE (Accelerated Solvent Extractor ASE 200 with Solvent Controller and AutoASE-Software (DIONEX)) immediately after freeze-drying.

Polar substances were extracted with approximately 10 mL Methanol/Water (80/20, v/v) at T=70° C. und p=140 bar, 5 min heating phase, 1 min static Extraction. Lipid substances were extracted with approximately 10 mL Methanol/Dichlormethan (40/60, v/v) at T=70° C. und p=140 bar, 5 min heating phase, 1 min static Extraction. Both extracts were collected in one extraction vial (Centrifuge tubes, 50 mL with screw-on lid and Septum for ASE (DIONEX)).

The following internal standards were added to the extracts: LC-Standards L-Methionine-d3, Boc-Ala-Gly-Gly-Gly-OH, L-Tryptophan-d5, Arginine 13C615N4, CoEnzyme Q1,2,4 and ribitol, L-glycine-2,2-d2. L-alanine-2,3,3,3-d4, alpha-methyl-glucopyranoside, nonadecanoic acid methyl ester, undecanoic acid methyl ester, tridecanoic acid, pentadecanoic acid, nonacosanoic acid. To the resulting mixture, 8 mL water-were added. The solid residues of plant and extraction thimble were discarded.

The extract was centrifuged at 1400 g for 5-10 minutes to speed-up phase-separation. For GC and LC analysis, 1 mL each was taken from the colourless methanol/water upper (polar) phase. The remaining upper phase was discarded. Of the dark-green, organic bottom phase 0.5 mL was taken for GC and LC analysis, respectively. All sample aliquots were evaporated using a IR-Dancer Infrared vacuum evaporator (Hettich), using a temperature maximum of 40° C. and a maximum pressure of 10 mbar.

e) LC/MS- and LC/MS/MS-Analysis

HPLC mobile phase was added to the lipid and polar residues, respectively (volume adjusted to the weighted sample) and an HPLC analysis using gradient elution was performed.

f) Derivatisation of the Lipid Phase for GC/MS-Analysis

For transmethanolysis, a mixture of 140 μl chloroform, 38 μL HCl (37% HCl in water), 320 μl methanol and 20 μl toluol was added to the residue. The sample container was carefully closed and reaction was carried out at 100° C. for 2 hours. Subsequently, the solution was evaporated and the pellet was dried completely.

The methoximation of carbonyl groups was achieved by a reaction with 100 μL methoxyamine-hydrochloride (5 mg/mL in Pyridine) for 1.5 hours at 60° C., in a closed vial. 20 μL of a mixture of linear, odd-numbered fatty acids was added to provide a time standard. Finally, derivatisation with 100 μL N-Methyl-N-(trimethylsilyl)-2,2,2-trifluoracetamide (MSTFA) took place in a closed vial for 30 minutes at 60° C. The final volume for GC injection was 220 μl.

g) Derivatisation of the Polar Phase for GC/MS-Analysis

The methoximation of carbonyl groups was achieved by a reaction with 50 μL methoxyamine-hydrochloride (5 mg/mL in Pyridine) for 1.5 hours at 60° C., in a closed vial. 10 μL of a mixture of linear, odd-numbered fatty acids was added to provide a time standard. Finally, derivatisation with 50 μL N-methyl-N-(trimethylsilyl)-2,2,2-trifluoracetamide (MSTFA) took place in a closed vial for 30 minutes at 60° C. The final volume for GC injection was 110

h) Analysis of Different Plant Samples

Samples were measured in sequences of 20. Each sequence contained 5 wild type and 5 transgenic plants grown under control-conditions, as well as 5 wild type and 5 transgenic plants from either the 4 day or 8 day drought treatment.

The peak height or peak area of each analyte (metabolite) was divided through the peak area of the respective internal standards. Data was normalized using the individual sample fresh weight. The resulting values were divided by the mean values found for wild type plants grown under control conditions and analysed in the same sequence, resulting in the so-called X-folds or ratios (see table 5), which represent values independent of the analytical sequence. These ratios indicate the behavior of the metabolite concentration of the target plants in comparison to the concentration in the wild type control plants.

All the sequence listings disclosed in this application are submitted on a compact disc (in duplicate) the content of which is incorporated in its entirety herein.

In table 5 the results of the metabolite screening for the plants transformed in gene F19K19.13 are shown

Tables 2-5:

TABLE 2
Duration of survival of transformed Arabidopsis thaliana after
imposition of drought stress on 3-week-old plants. Drought tolerance
was measured visually at daily intervals. Survival duration is the
average of all plants that survived longer than the wild type control.
The Maximum duration is the longest period that any single transformed
plant survived longer than the wild type control.

| SEQ | | | | |
|---|---|---|---|---|
| ID | | Plants | Average days of | Maximum days |
| No. | Gene | tested | survival after WT | of survival |
| | CONTROL | — | 0 | 0 |
| 1 | At1g61800 | 24 | 0.83 | 3 |
| 3 | At1g61810 | 24 | 0.83 | 3 |
| 5 | At5g65610 | 24 | 0.46 | 2 |
| 7 | At5g65600 | 24 | 0.46 | 2 |
| 9 | At2g22530 | 24 | 2.08 | 6 |
| 11 | At2g22540 | 24 | 2.08 | 6 |
| 13 | At3g57590 | 24 | 1.04 | 5 |
| 15 | At3g62170 | 24 | 1.88 | 7 |
| 17 | At3g62180 | 24 | 1.88 | 7 |
| 19 | At1g10120 | 24 | 1.21 | 4 |
| 21 | At1g10130 | 24 | 1.21 | 4 |
| 23 | At1g07710 | 11 | 2.72 | 6 |
| 25 | At1g07420 | 11 | 2.72 | 6 |
| 27 | At2g26890 | 45 | 2.33 | 6 |
| 29 | At2g35050 | 45 | 2.33 | 6 |
| 31 | At5g44860 | 45 | 2.33 | 6 |
| 33 | At5g44870 | 45 | 2.33 | 6 |
| 35 | At1g73490 | 24 | 0.88 | 5 |
| 37 | At1g73480 | 24 | 0.88 | 5 |
| 39 | At5g22400 | 100 | 1.6 | 4 |
| 41 | At5g22430 | 100 | 1.6 | 4 |
| 43 | At5g67210 | 100 | 1.6 | 4 |
| 45 | At5g67220 | 100 | 1.6 | 4 |
| 47 | At1g15820 | 11 | 1.82 | 4 |
| 49 | At1g15825 | 11 | 1.82 | 4 |
| 51 | At5g02470 | 19 | 0.95 | 3 |
| 53 | At5g02480 | 19 | 0.95 | 3 |
| 55 | At3g49120 | 25 | 0.6 | 3 |
| 57 | T2J13_50 | 25 | 0.6 | 3 |
| 59 | At2g25970 | 25 | 0.32 | 1 |
| 61 | dl4495c | 25 | 0.96 | 2 |
| 63 | dl4500c | 25 | 0.96 | 2 |
| 65 | At3g11170 | 25 | 0.76 | 2 |
| 67 | AT1G77310 | 25 | 0.76 | 2 |
| 69 | AT1G77320 | 25 | 0.76 | 2 |
| 71 | At2g20210 | 25 | 1.08 | 2 |
| 73 | At2g20200 | 25 | 1.08 | 2 |
| 75 | AT5g47370 | 25 | 1.08 | 2 |
| 77 | At4g33200 | 25 | 0.88 | 3 |
| 79 | At5g45340 | 25 | 0.88 | 3 |
| 81 | AT5G45810 | 25 | 0.72 | 2 |
| 83 | AT5G45820 | 25 | 0.72 | 2 |
| 85 | At2g02370 | 24 | 2.46 | 7 |
| 87 | AT5G39460 | 24 | 1.38 | 5 |
| 89 | MUL8.15 | 24 | 1.38 | 5 |
| 91 | F19K19.13 | 24 | 1.8 | 4 |
| | (At1g16540) | | | |
| 93 | MLP3.2 | 49 | 4.1 | 6 |
| | (At3g07575) | | | |
| 95 | F13K23.5 | 18 | 1.7 | 4 |
| | (At1g12800) | | | |
| 97 | MYJ24.7 | 24 | 1.0 | 4 |
| | (At5g23080) | | | |
| 99 | MBB18.23 | 24 | 1.1 | 3 |
| | (At5g38680) | | | |
| 101 | At2g28470 | 15 | 1.2 | 4 |
| 103 | F9F8.1 | 27 | 1.0 | 3 |
| | (At3g11210) | | | |
| 105 | F17C15_150 | 24 | 1.3 | 5 |
| | (At5g03730) | | | |
| 107 | At2g42690 | 24 | 0.9 | 4 |
| 109 | At4g31810 | 48 | 8.3 | 12 |
| 111 | At4g31820 | 48 | 8.3 | 12 |

TABLE 3
Photosynthetic yield as determined by chlorophyll florescence of transformed
Arabidopsis thaliana after imposition of drought stress on 3-week-old plants.
Measurements were taken at intervals after withholding water and reported as
photosynthetic yield (Y). Values are the average of 5 randomly selected plants.
These are compared to the MC24 strain for reference.

| | | Photosynthetic | | Photosynthetic | | Photosynthetic | |
|---|---|---|---|---|---|---|---|
| | | yield 6 days | | yield 10 days | | yield 14 days | |
| SEQ ID | | after final | MC24 | after final | MC24 | after final | MC24 |
| No. | Gene | watering | Reference | watering | Reference | watering | Reference |
| 1 | At1g61800 | 751 | 623 | 401 | 100 | | |
| 3 | At1g61810 | 751 | 623 | 401 | 100 | | |
| 5 | At5g65610 | 770 | 757 | 552 | 610 | | |
| 7 | At5g65600 | 770 | 757 | 552 | 610 | | |
| 9 | At2g22530 | 762 | 757 | 775 | 610 | 134 | 16 |
| 11 | At2g22540 | 762 | 757 | 775 | 610 | 134 | 16 |
| 13 | At3g57590 | 770 | 757 | 757 | 610 | | |
| 15 | At3g62170 | 766 | 757 | 734 | 610 | 152 | 16 |
| 17 | At3g62180 | 766 | 757 | 734 | 610 | 152 | 16 |
| 23 | At1g07710 | 779 | 757 | 752 | 610 | 305 | 16 |
| 25 | At1g07420 | 779 | 757 | 752 | 610 | 305 | 16 |
| 27 | At2g26890 | 779 | 765 | 730 | 413 | 292 | 54 |
| 29 | At2g35050 | 764 | 765 | 730 | 413 | 292 | 54 |
| 31 | At5g44860 | 764 | 765 | 730 | 413 | 292 | 54 |
| 33 | At5g44870 | 764 | 765 | 730 | 413 | 292 | 54 |
| 35 | At1g73490 | 764 | 314 | | | | |
| 37 | At1g73480 | 757 | 314 | | | | |
| 47 | At1g15820 | 757 | 757 | 536 | 610 | 148 | 16 |
| 49 | At1g15825 | 745 | 757 | 536 | 610 | 148 | 16 |
| 51 | At5g02470 | 745 | 783 | 290 | 210 | | |
| 53 | At5g02480 | 775 | 783 | 290 | 210 | | |
| 55 | At3g49120 | 775 | 783 | 265 | 210 | | |
| 57 | T2J13_50 | 787 | 783 | 265 | 210 | | |
| 59 | At2g25970 | 787 | 783 | 723 | 210 | | |
| 61 | dl4495c | 784 | 783 | 597 | 210 | | |
| 63 | dl4500c | 784 | 783 | 597 | 210 | | |
| 65 | At3g11170 | 784 | 783 | 666 | 210 | | |
| 67 | AT1G77310 | 763 | 783 | 666 | 210 | | |
| 69 | AT1G77320 | 763 | 783 | 666 | 210 | | |
| 71 | At2g20210 | 763 | 783 | 731 | 210 | | |
| 73 | At2g20200 | 762 | 783 | 731 | 210 | | |
| 75 | AT5g47370 | 762 | 783 | 731 | 210 | | |
| 77 | At4g33200 | 762 | 783 | 409 | 210 | | |
| 79 | At5g45340 | 792 | 783 | 409 | 210 | | |
| 81 | AT5G45810 | 792 | 783 | 585 | 210 | | |
| 83 | AT5G45820 | 766 | 783 | 585 | 210 | | |
| 85 | At2g02370 | 766 | 750 | 734 | 576 | 186 | 31 |
| 87 | AT5G39460 | 723 | 750 | 735 | 576 | 143 | 31 |
| 89 | MUL8.15 | 740 | 750 | 735 | 576 | 143 | 31 |
| 91 | F19K19.13 | 772 | 757 | 792 | 610 | | |
| 93 | MLP3.2 | 786 | 794 | 774 | 413 | 696 | 54 |
| 95 | F13K23.5 | 734 | 623 | 574 | 100 | | |
| 97 | MYJ24.7 | 775 | 757 | 699 | 610 | | |
| 99 | MBB18.23 | 767 | 757 | 748 | 610 | | |
| 101 | At2g28470 | 747 | 623 | 305 | 100 | | |
| 103 | F9F8.1 | 731 | 765 | 798 | 794 | 676 | 413 |
| 105 | F17C15_150 | 759 | 757 | 639 | 610 | 148 | 107 |
| 107 | At2g42690 | 736 | 623 | 206 | 100 | | |
| 109 | At4g31810 | 779 | 794 | 776 | 413 | 740 | 54 |
| 111 | At4g31820 | 779 | 794 | 776 | 413 | 740 | 54 |

TABLE 4
Details on PCR products used to identify the down-regulated genomic locus.

| SEQ | | Length | | Sequence | | | | Restriction enzyme |
|---|---|---|---|---|---|---|---|---|
| ID No. | Gene | PCR Product | | lenth | | Border | Method | or deg. Primer |
| 1 | At1g61800 | 700 | bp | 584 | bp | LB | Adaptor | MunI |
| 1 | At1g61800 | 700 | bp | 588 | bp | LB | Adaptor | MunI |
| 3 | At1g61810 | 700 | bp | 588 | bp | LB | Adaptor | MunI |
| 5 | At5g65610 | 1500 | bp | 603 | bp | LB | Adaptor | Psp1406I/Bsp119I |
| 5 | At5g65610 | 1000 | bp | 697 | bp | RB | Adaptor | BgIII |
| 5 | At5g65610 | 1000 | bp | 175 | bp | RB | Adaptor | BgIII |
| 7 | At5g65600 | 1000 | bp | 175 | bp | RB | Adaptor | BgIII |
| 9 | At2g22530 | 550 | bp | 465 | bp | LB | Adaptor | Psp1406I/Bsp119I |
| 9 | At2g22530 | 550 | bp | 444 | bp | LB | Adaptor | Psp1406I/Bsp119I |
| 11 | At2g22540 | 550 | bp | 444 | bp | LB | Adaptor | Psp1406I/Bsp119I |
| 13 | At3g57590 | 950 | bp | 842 | bp | LB | Adaptor | SpeI |
| 13 | At3g57590 | 900 | bp | 863 | bp | LB | Adaptor | SpeI |
| 15 | At3g62170 | 700 | bp | 450 | bp | LB | Adaptor | Psp1406I/Bsp119I |
| 15 | At3g62170 | 700 | bp | 419 | bp | LB | Adaptor | Psp1406I/Bsp119I |
| 18 | At3g62180 | 700 | bp | 450 | bp | LB | Adaptor | Psp1406I/Bsp119I |
| 19 | At1g10120 | 700 | bp | 161 | bp | LB | Adaptor | Psp1406I/Bsp119I |
| 19 | At1g10120 | 700 | bp | 161 | bp | LB | Adaptor | Psp1406I/Bsp119I |
| 19 | At1g10120 | 1400 | bp | 718 | bp | LB | Adaptor | SpeI |
| 21 | At1g10130 | 1400 | bp | 718 | bp | LB | Adaptor | SpeI |
| 23 | At1g07710 | 1300 | bp | 628 | bp | LB | Adaptor | MunI |
| 23 | At1g07710 | 1300 | bp | 678 | bp | LB | Adaptor | MunI |
| 25 | At1g07420 | 350 | bp | 251 | bp | RB | Adaptor | SpeI |
| 25 | At1g07420 | 350 | bp | 252 | bp | RB | Adaptor | SpeI |
| 27 | At2g26890 | 700 | bp | 615 | bp | LB | Adaptor | Psp1406I/Bsp119I |
| 27 | At2g26890 | 1200 | bp | 637 | bp | LB | Adaptor | MunI |
| 29 | At2g35050 | 500 | bp | 445 | bp | RB | Adaptor | Psp1406I/Bsp119I |
| 31 | At5g44860 | 400 | bp | 299 | bp | RB | Adaptor | BgIII |
| 31 | At5g44860 | 400 | bp | 297 | bp | RB | Adaptor | BgIII |
| 33 | At5g44870 | 400 | bp | 297 | bp | RB | Adaptor | BgIII |
| 35 | At1g73490 | 330 | bp | 244 | bp | LB | Adaptor | SpeI |
| 35 | At1g73490 | 650 | bp | 494 | bp | RB | Adaptor | MunI |
| 37 | At1g73480 | 650 | bp | 494 | bp | RB | Adaptor | MunI |
| 39 | At5g22400 | 850 | bp | 378 | bp | LB | Adaptor | BgIII |
| 41 | At5g22430 | 850 | bp | 378 | bp | LB | Adaptor | BgIII |
| 43 | At5g67210 | 900 | bp | 471 | bp | RB | TAIL | ADP3 |
| 43 | At5g67210 | 850 | bp | 471 | bp | RB | TAIL | ADP6 |
| 45 | At5g67220 | 850 | bp | 471 | bp | RB | TAIL | ADP6 |
| 47 | At1g15820 | 600 | bp | 456 | bp | LB | Adaptor | BgIII |
| 47 | At1g15820 | 600 | bp | 284 | bp | LB | Adaptor | BgIII |
| 49 | At1g15825 | 600 | bp | 456 | bp | LB | Adaptor | BgIII |
| 51 | At5g02470 | 2000 | bp | 170 | bp | RB | Adaptor | BgI II |
| 53 | At5g02480 | 2000 | bp | 170 | bp | RB | Adaptor | BgI II |
| 55 | At3g49120 | 950 | bp | 720 | bp | LB | Adaptor | Mun I |
| 57 | T2J13_50 | 950 | bp | 720 | bp | LB | Adaptor | Mun I |
| 59 | At2g25970 | 1100 | bp | 684 | bp | LB | Adaptor | Spe I |
| 61 | dl4495c | 580 | bp | 455 | bp | LB | Adaptor | Psp1406 I/Bsp119 I |
| 63 | dl4500c | 580 | bp | 455 | bp | LB | Adaptor | Psp1406 I/Bsp119 I |
| 65 | At3g11170 | 300 | bp | 207 | bp | LB | Adaptor | BgI II |
| 67 | AT1G77310 | 900 | bp | 401 | bp | LB | Tail | ADP8 |
| 69 | AT1G77320 | 900 | bp | 401 | bp | LB | Tail | ADP8 |
| 71 | At2g20210 | 460 | bp | 358 | bp | RB | Adaptor | Mun I |
| 73 | At2g20200 | 460 | bp | 358 | bp | RB | Adaptor | Mun I |
| 75 | AT5g47370 | 800 | bp | 607 | bp | LB | Adaptor | Mun I |
| 77 | At4g33200 | 800 | bp | 566 | bp | LB | Adaptor | BgI II |
| 79 | At5g45340 | 1200 | bp | 558 | bp | RB | Adaptor | BgI II |
| 81 | AT5G45810 | 800 | bp | 647 | bp | LB | Adaptor | BgI II |
| 83 | AT5G45820 | 800 | bp | 647 | bp | LB | Adaptor | BgI II |
| 85 | At2g02370 | 1300 | bp | 548 | bp | LB | Adaptor | Psp1406 I/Bsp 119 I |
| 87 | AT5G39460 | 700 | bp | 548 | bp | LB | Adaptor | BgI II |
| 89 | MUL8.15 | 700 | bp | 548 | bp | LB | Adaptor | BgI II |
| 91 | F19K19.13 | 400 | bp | 254 | bp | LB | Adaptor | BgIiI |
| 91 | F19K19.13 | 400 | bp | 256 | bp | LB | Adaptor | BgIII |
| 91 | F19K19.13 | 700 | bp | 625 | bp | RB | Adaptor | SpeI |
| 91 | F19K19.13 | 700 | bp | 479 | bp | RB | Adaptor | SpeI |
| 93 | MLP3.2 | 300 | bp | 212 | bp | LB | TAIL | ADP3 |
| 93 | MLP3.2 | 550 | bp | 419 | bp | RB | Adaptor | BgIII |
| 93 | MLP3.2 | 500 | bp | 362 | bp | RB | Adaptor | MunI |
| 93 | MLP3.2 | 450 | bp | 361 | bp | RB | Adaptor | MunI |
| 95 | F13K23.5 | 1600 | bp | 585 | bp | LB | Adaptor | MunI |
| 97 | MYJ24.7 | 550 | bp | 432 | bp | LB | Adaptor | BgIII |
| 99 | MBB18.23 | 1000 | bp | 458 | bp | LB | Adaptor | MunI |
| 99 | MBB18.23 | 1000 | bp | 723 | bp | LB | Adaptor | MunI |
| 99 | MBB18.23 | 550 | bp | 442 | bp | LB | Adaptor | Psp1406I/Bsp119I |
| 99 | MBB18.23 | 600 | bp | 450 | bp | LB | Adaptor | Psp1406I/Bsp119I |
| 101 | At2g28470 | 1100 | bp | 652 | | LB | Adaptor | Psp1406/Bsp119 |
| 103 | F9F8.1 | 1000 | bp | 518 | bp | LB | Adaptor | SpeI |
| 103 | F9F8.1 | 1000 | bp | 550 | bp | LB | Adaptor | SpeI |
| 105 | F17C15_150 | 850 | bp | 607 | bp | LB | Adaptor | BgIII |
| 105 | F17C15_150 | 1100 | bp | 402 | bp | LB | Adaptor | MunI |
| 107 | At2g42690 | 500 | bp | 444 | | LB | TAIL | ADP8 |
| 109 | At4g31810 | 1100 | bp | 590 | bp | LB | TAIL | ADP8 |
| 109 | At4g31810 | 650 | bp | 579 | bp | RB | TAIL | ADP8 |
| 111 | At4g31820 | 650 | bp | 579 | bp | RB | TAIL | ADP8 |

TABLE 5
Details on screening of metabolic activity (F19K19.13).

| | wild type | | | f19k19.13 | | |
|---|---|---|---|---|---|---|
| metabolite | control | 4 days | 8 days | control | 4 days | 8 days |
| 2,3-dimethyl-5-phytylquinol | 1.00 | 0.50 | 0.54 | | | 1.92 |
| 2-hydroxy-palmitic acid | 1.00 | 1.11 | 1.25 | 1.33 | 1.59 | 1.93 |
| 3,4-dihydroxyphenylalanine (=dopa) | 1.00 | 1.83 | 3.37 | | | |
| 3-hydroxy-palmitic acid | 1.00 | | | | | |
| 5-oxoproline | 1.00 | 1.56 | 1.81 | 2.42 | 2.89 | |
| alanine | 1.00 | 0.64 | 0.64 | 1.45 | 1.25 | |
| alpha linolenic acid (c18:3 (c9, c12, c15)) | 1.00 | 1.24 | 1.40 | 1.27 | 1.65 | 1.85 |
| alpha-tocopherol | 1.00 | 1.05 | 1.14 | | | |
| aminoadipic acid | 1.00 | 1.73 | 1.65 | | | |
| anhydroglucose | 1.00 | 1.02 | 1.16 | 1.42 | | |
| arginine | 1.00 | 0.39 | 0.33 | | 0.85 | |
| aspartic acid | 1.00 | 2.72 | 2.96 | 2.94 | 4.27 | 10.08 |
| beta-apo-8′ carotenal | 1.00 | 1.21 | 1.22 | | | |
| beta-carotene | 1.00 | 1.25 | 1.26 | | | 1.91 |
| beta-sitosterol | 1.00 | 1.39 | 1.51 | | | 1.82 |
| beta-tocopherol | 1.00 | 0.54 | 0.60 | | | 3.13 |
| palmitic acid | 1.00 | 1.07 | 1.25 | | | |
| (delta-7-cis,10-cis)-hexadecadienic acid | 1.00 | 1.01 | 1.04 | | | |
| (c16:2 me) | | | | | | |
| hexadecatrienic acid (c16:3 me) | 1.00 | 1.19 | 1.29 | | 1.46 | 1.57 |
| margaric acid (c17:0 me) | 1.00 | 1.19 | 1.28 | 1.24 | | 1.79 |
| delta-15-cis-tetracosenic acid (c24:1 me) | 1.00 | 1.16 | 1.34 | 1.13 | 1.71 | 1.81 |
| campesterol | 1.00 | 1.32 | 1.65 | 1.75 | 2.46 | 3.21 |
| cerotic acid (c26:0) | 1.00 | 0.74 | 1.00 | 0.38 | | |
| citrulline | 1.00 | 0.51 | 0.33 | 1.88 | 1.70 | |
| cryptoxanthine | 1.00 | 1.00 | 0.90 | | | |
| eicosenoic acid (20:1) | 1.00 | 0.81 | 0.81 | | | |
| ferulic acid | 1.00 | 1.37 | 1.47 | 1.66 | 2.62 | |
| fructose | 1.00 | 14.10 | 19.78 | | | |
| fumarat | 1.00 | 5.19 | 9.33 | | | |
| galactose | 1.00 | 1.29 | 1.42 | | | |
| g-aminobutyric acid | 1.00 | 1.16 | 1.32 | 1.62 | 2.21 | 5.00 |
| gamma - tocopherol | 1.00 | 0.54 | 0.60 | | | 1.69 |
| gluconic acid | 1.00 | 2.24 | 3.08 | | | |
| glucose | 1.00 | 14.97 | 20.73 | | | |
| glutamine | 1.00 | 0.98 | 1.17 | 2.88 | 4.90 | |
| glutamic acid | 1.00 | 2.01 | 2.69 | 2.49 | 6.81 | |
| glycerate | 1.00 | 1.87 | 2.04 | 0.85 | 1.21 | |
| glycerinaldehyd | 1.00 | 1.11 | 1.12 | 1.56 | 2.59 | 4.80 |
| glycerol | 1.00 | 1.22 | 1.29 | | | |
| glycerol-3-phosphate | 1.00 | 1.86 | 2.41 | 1.90 | | |
| glycine | 1.00 | 0.25 | 0.26 | | | |
| homoserine | 1.00 | 0.61 | 0.69 | | | |
| inositol | 1.00 | 4.19 | 6.32 | 2.02 | 5.03 | 21.68 |
| isoleucine | 1.00 | 1.28 | 1.66 | 1.44 | 1.96 | 8.02 |
| iso-maltose | 1.00 | 2.63 | 3.02 | | | |
| isopentenyl pyrophosphate | 1.00 | 1.80 | 2.62 | 1.79 | 3.48 | |
| leucine | 1.00 | 1.34 | 1.74 | 1.59 | 2.33 | 6.58 |
| lignoceric acid (c24:0) | 1.00 | 0.91 | 1.02 | | | |
| linoleic acid (c18:2 (c9, c12)) | 1.00 | 1.19 | 1.38 | | 1.41 | 1.66 |
| luteine | 1.00 | 1.26 | 1.33 | | | |
| lycopene | | | | | 1.68 | 1.84 |
| malate | 1.00 | 2.91 | 3.46 | 1.72 | | |
| mannose | 1.00 | 16.40 | 17.80 | 2.56 | | 79.91 |
| melissinsäure | 1.00 | 1.26 | 1.23 | | | |
| methionine | 1.00 | 1.13 | 1.12 | 1.64 | | |
| methylgalactofuranoside | 1.00 | 1.24 | 1.49 | | 1.68 | 1.84 |
| methylgalactopyranoside | 1.00 | 1.25 | 1.36 | | 1.37 | 1.70 |
| palmitic acid (c16:0) | 1.00 | 1.16 | 1.38 | 1.23 | 1.61 | 1.98 |
| phenylalanine | 1.00 | 0.92 | 1.10 | | | |
| phosphate | 1.00 | 2.15 | 2.73 | 2.37 | 7.31 | 11.38 |
| proline | 1.00 | 1.23 | 4.77 | 2.23 | 4.26 | |
| putrescine | 1.00 | | 0.39 | | | |
| pyruvate | 1.00 | 1.30 | 1.21 | | | |
| raffinose | 1.00 | 17.04 | 74.78 | | 7.06 | |
| ribonsäure | 1.00 | 2.32 | 3.43 | | | |
| serine | 1.00 | 0.98 | 1.13 | 1.47 | 1.68 | |
| shikimate | 1.00 | 1.11 | 1.07 | 0.85 | 0.79 | 0.71 |
| sinapine acid | 1.00 | 2.74 | 3.44 | 1.30 | 1.87 | |
| stearic acid (c18:0) | 1.00 | 1.18 | 1.35 | 1.16 | 1.36 | 1.79 |
| succinate | 1.00 | 2.98 | 3.49 | 2.01 | | |
| sucrose | 1.00 | 1.42 | 1.70 | 1.46 | 2.60 | 3.87 |
| threonine | 1.00 | 1.26 | 1.68 | 3.28 | 4.06 | |
| tryptophane | 1.00 | 1.13 | 1.89 | | 1.86 | |
| tyrosine | 1.00 | 1.56 | 2.01 | 1.46 | | |
| ubichinone | 1.00 | 1.02 | 1.20 | | | 3.13 |
| udp-glucose | 1.00 | 1.53 | 1.94 | 2.02 | 4.85 | |
| valine | 1.00 | 0.98 | 1.18 | 1.51 | 1.75 | 3.18 |
| zeaxanthine | 1.00 | 1.27 | 1.34 | | | |

1. A transformed plant cell with altered metabolic activity compared to a corresponding non transformed wild type plant cell, wherein the metabolic activity is altered by an inactivated or down-regulated gene and results in increased tolerance and/or resistance to an environmental stress as compared to a corresponding non-transformed wild type plant cell.

2. The transformed plant cell of claim 1, wherein metabolic activity is altered by one or more inactivated or down-regulated genes encoded by one or more nucleic acid sequences selected from the group consisting of:
a) nucleic acid molecule encoding the polypeptide shown in SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280
b) nucleic acid molecule comprising the nucleic acid molecule shown in SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or
116, 130, 132, 146, 162, 177, 210, 212, 220, 222, 242, and/or 279
c) nucleic acid molecule comprising a nucleic acid sequence, which, as a result of the degeneracy of the genetic code, can be derived from a polypeptide sequence depicted in SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280
d) nucleic acid molecule encoding a polypeptide having at least 50% identity with the amino acid sequence of the polypeptide encoded by the nucleic acid molecule of (a) to (c) and having the biological activity represented by protein of SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280
e) nucleic acid molecule encoding a polypeptide which is isolated with the aid of monoclonal antibodies against a polypeptide encoded by one of the nucleic acid molecules of (a) to (d) and having the biological activity represented by the protein of SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280
f) nucleic acid molecule which is obtainable by screening a suitable nucleic acid library under stringent hybridization conditions with a probe comprising one of the sequences of the nucleic acid molecule of (a) or (b) or with a fragment thereof having at least 15 nucleotides of the nucleic acid molecule characterized in (a) to (c) and encoding a polypeptide having the biological activity represented by a protein whose reduction or deletion results in increased tolerance and/or resistance to an environmental stress
or which comprises a sequence which is complementary thereto.

3. The transformed plant cell of claim 2, wherein the nucleic acid is at least about 50% homologous to said sequences of SEQ ID NO 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or 116, 130, 132, 146, 162, 177, 210, 212, 220, 222, 242, and/or 279.

4. The transformed plant cell of claim 1 derived from a monocotyledonous plant.

5. The transformed plant cell of claim 1 derived from a dicotyledonous plant.

6. The transformed plant cell of claim 1, wherein the plant is selected from the group comprised of maize, wheat, rye, oat, triticale, rice, barley, soybean, peanut, cotton, rapeseed, canola, manihot, pepper, sunflower, flax, borage, safflower, linseed, primrose, rapeseed, turnip rape, tagetes, solanaceous plants, potato, tobacco, eggplant, tomato, Vicia species, pea, alfalfa, coffee, cacao, tea, Salix species, oil palm, coconut, perennial grass, forage crops and Arabidopsis thaliana.

7. The transformed plant cell of claim 1, derived from a gymnosperm plant.

8. The transformed plant cell of claim 1, wherein the plant is selected from the group of spruce, pine and fir.

9. A transformed plant generated from a plant cell according to claim 1 and which is a monocot or dicot plant.

10. A transformed plant generated from a plant cell according to claim 1 and which is a gymnosperm plant.

11. A seed produced by the transformed plant of claim 9, wherein the seed is at least genetically heterozygous for a gene that when inactivated or down-regulated confers increased tolerance to environmental stress as compared to a wild type plant.

12. A method of producing a transformed plant with altered metabolic activity compared to a corresponding non-transformed wild type plant cell by inactivation or down-regulation of a gene in the transformed plant resulting in increased tolerance and/or resistance to environmental stress as compared to a corresponding non-transformed wild type plant, comprising
(a) transforming a plant cell by inactivation or down-regulation of one or more genes, and
(b) generating from the plant cell a transformed plant with an increased tolerance and/or resistance to environmental stress as compared to a corresponding wild type plant.

13. A method of inducing increased tolerance and/or resistance to environmental stress as compared to a corresponding non-transformed wild type plant in a plant cell of the claim 1 by altering metabolic activity compared to a corresponding non-transformed wild type plant cell by inactivation or down-regulation of one or more genes encoded by one or more nucleic acids selected from a group consisting of sequences of SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or
114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or 116, 130, 132, 146, 162, 177, 210, 212, 220, 222, 242, and/or 279
and/or homologs thereof.

14. The method of claim 13, wherein the gene encoding nucleic acid is at least about 50% homologous to sequences of SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or 116, 130, 132, 146, 162, 177, 210, 212, 220, 222, 242, and/or 279.

15. The method of the claim 12, wherein the inactivation or down-regulation of said gene is achieved by double-stranded RNA interference (dsRNAi), introduction of an antisense nucleic acid, a ribozyme, an antisense nucleic acid combined with a ribozyme, a nucleic acid encoding a co-suppressor, a nucleic acid encoding a dominant negative protein, DNA- or RNA- or protein-binding factors targeting said gene or -RNA or -proteins, RNA degradation inducing viral nucleic acids and expression systems, systems for inducing a homolog recombination of said genes, mutations in said genes or a combination of the above.

16. A plant expression cassette comprising a nucleic acid construct, which when expressed allows inactivation or down-regulation of one or more genes encoded by one or more nucleic acids selected from the group consisting of sequences of SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 11, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or 116; 130, 132, 146, 162, 177, 210, 212, 220, 222, 242, and/or 279 and/or homologs thereof and/or parts thereof by a method of claim 15.

17. A transformed plant cell with an inactivated or down-regulated gene encoded by a nucleic acid sequence selected from the group consisting of sequences of SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or 116, 130, 132, 146, 162, 177, 210, 212, 220, 222, 242, and/or 279 and/or homologs thereof.

18. An isolated nucleic acid molecule which comprises a nucleic acid molecule selected from the group consisting of:
a) nucleic acid molecule which encodes a polypeptide comprising the polypeptide shown in 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or
115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280;
b) nucleic acid molecule which comprising the polynucleotide shown in SEQ ID NO 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or
116, 130, 132, 146, 162, 177, 210, 212, 220, 222, 242, and/or 279;
c) nucleic acid molecule comprising a nucleic acid sequence, which, as a result of the degeneracy of the genetic code, can be derived from a polypeptide sequence depicted (b) and having the biological activity represented by protein of SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280;
d) nucleic acid molecule encoding a polypeptide having at least 50% identity with the amino acid sequence of the polypeptide encoded by the nucleic acid molecule of (a) or (c) and having a biological activity represented by protein of SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or 92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or 117, 131, 133, 147, 163, 178, 211, 213, 221, 223, 243 and/or 280;
e) nucleic acid molecule encoding a polypeptide, which is isolated with the aid of monoclonal antibodies against a polypeptide encoded by one of the nucleic acid molecules of (a) to (c) and having a biological activity represented by the peptides shown in (a);
f) nucleic acid molecule which is obtainable by screening a suitable library under stringent hybridization conditions wits a probe comprising one of the sequences of the nucleic acid molecule of (a) to (c) or with a fragment of at least 15 nucleotide characterized in (a) to (c) and encoding a polypeptide having the biological activity represented the peptides shown in (a);
g) a nucleic acid molecule having at least 70% sequence identity to polynucleotide selected from the groups consisting of the polynucleotides shown in SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or
116, 130, 132, 146, 162, 177, 210, 212, 220, 222, 242, and/or 279;
or which comprises a sequence which is complementary thereto; whereby the nucleic acid molecule according to (a) to (g) is at least in one or more nucleotides different from the sequence depicted in SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 1.24, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or 116, 130, 132, 146, 162, 177, 210, 212, 220, 222, 242, and/or 279
and which encodes a protein which differs at least in one or more amino acids from the protein sequences depicted in SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 1.23, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or 117, 131, 133, 147, 163, 178, 211, 213, 221, 223, 243 and/or 280.

19. An isolated polypeptide encoded by a nucleic acid molecule as claimed in claim 18.

20. An antibody, which specifically binds to the polypeptide as claimed in claim 19.

21. A transformed plant cell wherein increased tolerance and/or resistance to an environmental stress is conferred by one or more inactivated or down-regulated genes encoded by one or more nucleic acid sequences selected from the group consisting of:
a) nucleic acid molecule encoding the polypeptide shown in SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280;
b) nucleic acid molecule comprising the nucleic acid molecule shown in SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or
116, 130, 132, 146, 162, 177, 210, 212, 220, 222, 242, and/or 279;
c) nucleic acid molecule comprising a nucleic acid sequence, which, as a result of the degeneracy of the genetic code, can be derived from a polypeptide sequence depicted in SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280;
d) nucleic acid molecule encoding a polypeptide having at least 50% identity with the amino acid sequence of the polypeptide encoded by the nucleic acid molecule of (a) to (c) and having the biological activity represented by protein of SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280;
e) nucleic acid molecule encoding a polypeptide which is isolated with the aid of monoclonal antibodies against a polypeptide encoded by one of the nucleic acid molecules of (a) to (d) and having the biological activity represented by the protein of SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280;
f) nucleic acid molecule which is obtainable by screening a suitable nucleic acid library under stringent hybridization conditions with a probe comprising one of the sequences of the nucleic acid molecule of (a) or (b) or with a fragment thereof having at least 15 nucleotide of the nucleic acid molecule characterized in (a) to (c) and encoding a polypeptide having the biological activity represented by protein whose reduction or deletion results in increased tolerance and/or resistance to an environmental stress; and
g) a nucleic acid molecule having at least 70% sequence identity to polynucleotide selected from the groups consisting of the polynucleotides shown in SEQ ID NO
2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 and/or 90 and/or
92, 94, 96, 98, 100, 102, 104, 106, 108, 110 and/or 112 and/or 115, 119, 121, 123, 125, 127, 129, 135, 137, 139, 141, 143, 145, 149, 151, 153, 155, 157, 159, 161, 165, 167, 169, 174, 176, 179, 181, 183, 185, 187, 189, 191, 193, 195, 197, 199, 201, 203, 205, 207, 209, 215, 217, 219, 225, 227, 229, 231, 233, 235, 237, 239, 241, 245, 247, 249, 251, 253, 255, 257, 259, 261, 263, 265, 267, 269, 271, 273, 274, 276, 278, 281 and/or 283 and/or
117, 131, 133, 147, 163, 178, 211, 213, 221, 223, 243 and/or 280;
or which comprises a sequence which is complementary thereto.

22. A plant comprising a cell of claim 21.

23. The method of claim 12 wherein the inactivated or down-regulated gene is encoded by one or more nucleic acids selected from a group consisting of sequences of SEQ ID NO
1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87 and/or 89 and/or
91, 93, 95, 97, 99, 101, 103, 105, 107, 109 and/or 111 and/or 114, 118, 120, 122, 124, 126, 128, 134, 136, 138, 140, 142, 144, 148, 150, 152, 154, 156, 158, 160, 164, 166, 168, 170, 171, 172, 173, 175, 180, 182, 184, 186, 188, 190, 192, 194, 196, 198, 200, 202, 204, 206, 208, 214, 216, 218, 224, 226, 228, 230, 232, 234, 236, 238, 240, 244, 246, 248, 250, 252, 254, 256, 258, 260, 262, 264, 266, 268, 270, 272, 275, 277 and/or 282 and/or
116, 130, 132, 146, 162, 177, 210, 212, 220, 222, 242, and/or 279
and/or homologs thereof.
